(19) United States
(12) Patent Application Publication
(10) Pub. No.: US 2006/0019408 A1
(43) Pub. Date: Jan. 26, 2006

(54) Optical biosensors and methods of use thereof

(75) Inventors: Alan S. Waggoner, Pittsburgh, PA (US); Bruce A. Armitage, Pittsburgh, PA (US); William E. Brown, Pittsburgh, PA (US)

Correspondence Address: MORGAN, LEWIS & BOCKIUS LLP, 1701 MARKET STREET, PHILADELPHIA, PA, 19103-2921, US

(73) Assignee: Carnegie Mellon University

(21) Appl. No.: 11/077,999

(22) Filed: Mar. 11, 2005

Related U.S. Application Data
(63) Continuation of application No. PCT/US03/29289, filed on Sep. 15, 2003.
(60) Provisional application No. 60/410,834, filed on Sep. 13, 2002.

(51) Int. Cl. G01N 33/543

(52) U.S. Cl. 436/518

(57) ABSTRACT

A fundamental biosensor for detection of biological or environmental analytes is provided. The biosensor comprises a selectivity component for recognition of a target molecule and a reporter molecule that is sensitive to changes in the microenvironment. Methods of using the biosensor are also provided, including in vivo and in vitro applications using biosensor molecules that optionally may be attached to a surface.

### BACKGROUND

The identification, analysis and monitoring of biological analytes (such as polypeptides, polynucleotides, polysaccharides and the like) or environmental analytes (such as pesticides, bio-warfare agents, food contaminants and the like) has become increasingly important for research and industrial applications. Conventionally, analyte detection systems are based on analyte-specific binding between an analyte and an analyte-binding receptor. Such systems typically require complex multicomponent detection systems (such as ELISA sandwich assays) or electrochemical detection systems, or require that both the analyte and the receptor are labeled with detection molecules (for example fluorescence resonance energy transfer or FRET systems).

One method for detecting analyte-binding agent interactions involves a solid phase format employing a reporter labeled analyte-binding agent whose binding to or release from a solid surface is dependent on the presence of analyte. In a typical solid-phase sandwich type assay, for example, the analyte to be measured is an analyte with two or more binding sites, allowing analyte binding both to a receptor carried on a solid surface, and to a reporter-labeled second receptor. The presence of analyte is detected based on the presence of the reporter bound to the solid surface.

A variety of devices for detecting analyte/receptor interactions are also known. The most basic of these are purely chemical/enzymatic assays in which the presence or amount of analyte is detected by measuring or quantitating a detectable reaction product, such as gold immunoparticles. Analyte/receptor interactions can also be detected and quantitated by radiolabel assays. Quantitative binding assays of this type involve two separate components: a reaction substrate, e.g., a solid-phase test strip and a separate reader or detector device, such as a scintillation counter or spectrophotometer. The substrate is generally unsuited to multiple assays, or to miniaturization, for handling multiple analyte assays from a small amount of body-fluid sample.

Biosensor devices integrate the assay substrate and detector surface into a single device. One general type of biosensor employs an electrode surface in combination with current or impedance measuring elements for detecting a change in current or impedance in response to the presence of a ligand-receptor binding event. This type of biosensor is disclosed, for example, in U.S. Pat. No. 5,567,301.

Gravimetric biosensors employ a piezoelectric crystal to generate a surface acoustic wave whose frequency, wavelength and/or resonance state are sensitive to surface mass on the crystal surface. The shift in acoustic wave properties is therefore indicative of a change in surface mass, e.g., due to a ligand-receptor binding event. U.S. Pat. Nos. 5,478,756 and 4,789,804 describe gravimetric biosensors of this type.

Biosensors based on surface plasmon resonance (SPR) effects have also been proposed, for example, in U.S. Pat. Nos. 5,485,277 and 5,492,840. These devices exploit the shift in SPR surface reflection angle that occurs with perturbations, e.g., binding events, at the SPR interface. Finally, a variety of biosensors that utilize changes in optical properties at a biosensor surface are known, e.g., U.S. Pat. No. 5,268,305.

All of the above analyte detection systems are characterized by the requirement for a secondary detection system to monitor interactions between the analyte and the receptor. A need still exists for a direct, homogeneous assay for analyte detection which will be more versatile in terms of the range of applications and devices with which it can be used.

### SUMMARY

This application provides biosensors, compositions of biosensors and methods of use thereof.

In one aspect, the application provides a biosensor comprising a selectivity component and at least one reporter molecule, wherein binding of the selectivity component to a target molecule produces a detectable change in the signal of the reporter molecule.

In various embodiments, the selectivity component may be selected from the group consisting of a monoclonal antibody, polyclonal antibody, Fv fragment, single chain Fv (scFv) fragment, Fab′ fragment, F(ab′)2 fragment, single domain antibody, camelized antibody, humanized antibody, diabodies, tribodies, tetrabodies, aptamer, and template imprinted material. In various embodiments, the reporter molecule is responsive to environmental changes, including, for example, pH sensitive molecules, restriction sensitive molecules, polarity sensitive molecules, and mobility sensitive molecules. The reporter molecule may be either fluorescent or chemiluminescent. In certain embodiments, the reporter molecule may be associated with the selectivity component proximal to a region that binds to the target molecule. In an exemplary embodiment, the reporter molecule is covalently attached to the selectivity component proximal to a region that binds to the target molecule. The biosensor may respond to changes in the concentration of the target molecule and may be useful for monitoring the concentration of a target molecule over time.

In certain embodiments, the biosensor may comprise two or more reporter molecules, which may be the same or different reporter molecules. The reporter molecule may be detectable by a variety of methods, including, for example, a fluorescent spectrometer, filter fluorometer, microarray reader, optical fiber sensor reader, epifluorescence microscope, confocal laser scanning microscope, two photon excitation microscope, or a flow cytometer. In an exemplary embodiment, the reporter molecule is detectable through tissue.

In certain embodiments, the reporter molecule may be represented by structure I: 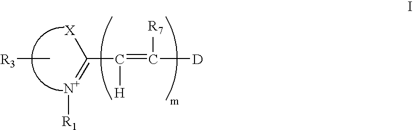
wherein:
the curved lines represent the atoms necessary to complete a structure selected from one ring, two fused rings, and three fused rings, each said ring having five or six atoms, and each said ring comprising carbon atoms and, optionally, no more than two atoms selected from oxygen, nitrogen and sulfur;
D is 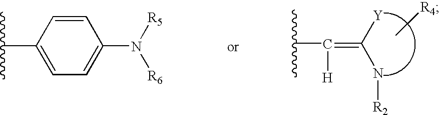
m is 1,2, 3 or 4;
X and Y are independently selected from the group consisting of O, S, and —C(CH₃)₂—;
at least one R₁, R₂, R₃, R₄, R₅, R₆, or R₇ is a reactive group selected from the group consisting of isothiocyanate, isocyanate, monochlorotriazine, dichlorotriazine, mono- or di-halogen substituted pyridine, mono- or di-halogen substituted diazine, phosphoramidite, maleimide, aziridine, sulfonyl halide, acid halide, hydroxysuccinimide ester, hydroxysulfosuccinimide ester, imido ester, hydrazine, axidonitrophenyl, azide, 3-(2-pyridyl dithio)-proprionamide, glyoxal, haloacetamido, and aldehyde;
providing that when any of R₁, R₂, R₃, R₄, R₅, R₆, or R₇ is not a reactive group it is selected from the group consisting of H, alkyl, aryl, and an -E-F group;
wherein:

F is selected from the group consisting of hydroxy, protected hydroxy, alkoxy, sulfonate, sulfate, carboxylate, and lower alkyl substituted amino or quartenary amino;
E is spacer group of formula —(CH₂)ₙ— wherein n is an integer from 0-5 inclusively;
further providing that R₁ and R₂ may be joined by a —CHR₈-CHR₈— or —BF₂-biradical;
   wherein;
R₈ independently for each occurrence is selected from the group consisting of hydrogen, amino, quaternary amino, aldehyde, aryl, hydroxyl, phosphoryl, sulfhydryl, water solubilizing groups, alkyl groups of twenty-six carbons or less, lipid solubilizing groups, hydrocarbon solubilizing groups, groups promoting solubility in polar solvents, groups promoting solubility in nonpolar solvents, and -E-F; and
further providing that any of R₁, R₂, R₃, R₄, R₅, R₆, or R₇ may be substituted with halo, nitro, cyano, —CO₂alkyl, —CO₂H, —CO₂aryl, NO₂, or alkoxy.
   In other embodiments, the reporter molecule may be represented by structure II: 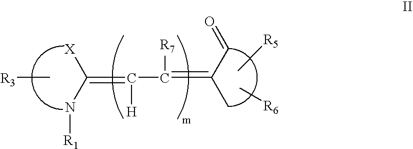
   wherein:
the curved lines represent the atoms necessary to complete a structure selected from one ring, two fused rings, and three fused rings, each said ring having five or six atoms, and each said ring comprising carbon atoms and, optionally, no more than two atoms selected from oxygen, nitrogen and sulfur;
D is 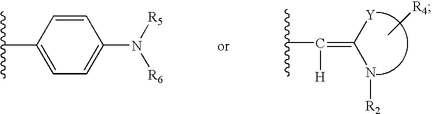
m is 1, 2, 3 or 4;
X and Y are independently selected from the group consisting of O, S, and —C(CH₃)₂—;
at least one R₁, R₂, R₃, R₄, R₅, R₆, or R₇ is a reactive group selected from the group consisting of isothiocyanate, isocyanate, monochlorotriazine, dichlorotriazine, mono- or di-halogen substituted pyridine, mono- or di-halogen substituted diazine, phosphoramidite, maleimide, aziridine, sulfonyl halide, acid halide, hydroxysuccinimide ester, hydroxysulfosuccinimide ester, imido ester, hydrazine, axidonitrophenyl, azide, 3-(2-pyridyl dithio)-proprionamide, glyoxal, haloacetamido, and aldehyde;
providing that when any of R₁, R₂, R₃, R₄, R₅, R₆, or R₇ is not a reactive group it is selected from the group consisting of H, alkyl, aryl, and an -E-F group;
wherein:
   F is selected from the group consisting of hydroxy, protected hydroxy, alkoxy, sulfonate, sulfate, carboxylate, and lower alkyl substituted amino or quartenary amino;
   E is spacer group of formula —(CH₂)ₙ— wherein n is an integer from 0-5 inclusively;
   further providing that R₁ and R₂ may be joined by a —CHR₈-CHR₈— or —BF₂-biradical;
wherein;
   R₈ independently for each occurrence is selected from the group consisting of hydrogen, amino, quaternary amino, aldehyde, aryl, hydroxyl, phosphoryl, sulfhydryl, water solubilizing groups, alkyl groups of twenty-six carbons or less, lipid solubilizing groups, hydrocarbon solubilizing groups, groups promoting solubility in polar solvents, groups promoting solubility in nonpolar solvents, and -E-F; and
   further providing that any of R₁, R₂, R₃, R₄, R₅, R₆, or R₇ may be substituted with halo, nitro, cyano, —CO₂alkyl, —CO₂H, —CO₂aryl, NO₂, or alkoxy.

In other embodiments, the reporter molecule may be represented by structure III: 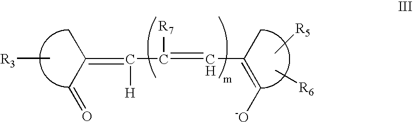
wherein:
the curved lines represent the atoms necessary to complete a structure selected from one ring, two fused rings, and three fused rings, each said ring having five or six atoms, and each said ring comprising carbon atoms and, optionally, no more than two atoms selected from oxygen, nitrogen and sulfur;
D is 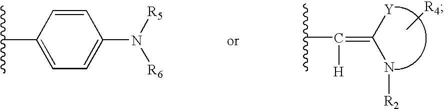
m is 1, 2, 3 or 4;
X and Y are independently selected from the group consisting of O, S, and —C(CH₃)₂—;
at least one R₁, R₂, R₃, R₄, R₅, R₆, or R₇ is a reactive group selected from the group consisting of isothiocyanate, isocyanate, monochlorotriazine, dichlorotriazine, mono- or di-halogen substituted pyridine, mono- or di-halogen substituted diazine, phosphoramidite, maleimide, aziridine, sulfonyl halide, acid halide, hydroxysuccinimide ester, hydroxysulfosuccinimide ester, imido ester, hydrazine, axidonitrophenyl, azide, 3-(2-pyridyl dithio)-proprionamide, glyoxal, haloacetamido, and aldehyde;
providing that when any of R₁, R₂, R₃, R₄, R₅, R₆, or R₇ is not a reactive group it is selected from the group consisting of H, alkyl, aryl, and an -E-F group;
wherein:
   F is selected from the group consisting of hydroxy, protected hydroxy, alkoxy, sulfonate, sulfate, carboxylate, and lower alkyl substituted amino or quartenary amino;
   E is spacer group of formula —(CH₂)ₙ— wherein n is an integer from 0-5 inclusively;
further providing that R₁ and R₂ may be joined by a —CHR₈—CHR₈— or —BF₂-biradical;
wherein;
   R₈ independently for each occurrence is selected from the group consisting of hydrogen, amino, quaternary amino, aldehyde, aryl, hydroxyl, phosphoryl, sulfhydryl, water solubilizing groups, alkyl groups of twenty-six carbons or less, lipid solubilizing groups, hydrocarbon solubilizing groups, groups promoting solubility in polar solvents, groups promoting solubility in nonpolar solvents, and -E-F; and
   further providing that any of R₁, R₂, R₃, R₄, R₅, R₆, or R₇ may be substituted with halo, nitro, cyano, —CO₂alkyl, —CO₂H, —CO₂aryl, NO₂, or alkoxy.
      In another embodiment, the reporter molecule may be represented by structure IV: 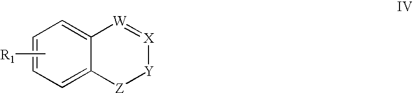
      wherein:
W is N or C(R₁);
X is C(R₂)₂;
Y is C(R₃)₂;
Z is NR₁, O, or S;
at least one R₁, R₂, or R₃ is a reactive group selected from the group consisting of isothiocyanate, isocyanate, monochlorotriazine, dichlorotriazine, mono- or di-halogen substituted pyridine, mono- or di-halogen substituted diazine, phosphoramidite, maleimide, aziridine, sulfonyl halide, acid halide, hydroxysuccinimide ester, hydroxysulfosuccinimide ester, imido ester, hydrazine, axidonitrophenyl, azide, 3-(2-pyridyl dithio)-proprionamide, glyoxal and aldehyde;
providing that when any of R₁, R₂, or R₃ is not a reactive group it is selected from the group consisting of H; alkyl; aryl; 1, 2, or 3 fused rings, each said ring having five or six atoms, and each said ring comprising carbon atoms and, optionally, no more than two atoms selected from oxygen, nitrogen and sulfur; and an -E-F group;
wherein:
   F is selected from the group consisting of hydroxy, protected hydroxy, alkoxy, sulfonate, sulfate, carboxylate, and lower alkyl substituted amino or quartenary amino;
   E is spacer group of formula —(CH₂)ₙ— wherein n is an integer from 0-5 inclusively;
   further providing that two R₃ taken together may form O, S, NR₁, or N⁺(R₁)₂; or two R₃ along with R₂ may form 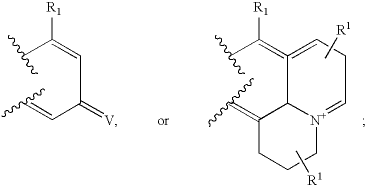
   wherein V is O, S, NR₁, or N⁺(R₁)₂; and
   further providing that any of R₁, R₂, or R₃ may be substituted with halo, nitro, cyano, —CO₂alkyl, —CO₂H, —CO₂aryl, NO₂, or alkoxy.

In other embodiments, the reporter molecule may be represented by structure V: 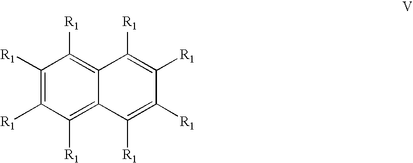
wherein:
at least one R₁ is a reactive group selected from the group consisting of isothiocyanate, isocyanate, monochlorotriazine, dichlorotriazine, mono- or di-halogen substituted pyridine, mono- or di-halogen substituted diazine, phosphoramidite, maleimide, aziridine, sulfonyl halide, acid halide, hydroxysuccinimide ester, hydroxysulfosuccinimide ester, imido ester, hydrazine, axidonitrophenyl, azide, 3-(2-pyridyl dithio)-proprionamide, glyoxal, haloacetamido, and aldehyde;
providing that when any of R₁ is not a reactive group it is selected from the group consisting of H, alkyl, aryl, and an -E-F group;
wherein:
   F is selected from the group consisting of hydroxy, protected hydroxy, alkoxy, sulfonate, sulfate, carboxylate, and lower alkyl substituted amino or quartenary amino;
   E is spacer group of formula —(CH₂)ₙ— wherein n is an integer from 0-5 inclusively;
further providing that any two adjacent R₁ may be joined to form a fused aromatic ring; and
further providing that R₁ may be substituted with halo, nitro, cyano, —CO₂alkyl, —CO₂H, —CO₂aryl, NO₂, or alkoxy.

In exemplary embodiments, the reporter molecule may be restriction sensor dye such as a monomethine cyanine dye or a trimethine cyanine dye.

In other embodiments, the biosensor may further comprise a chemical handle. The chemical handle may be used to facilitate isolation, immobilization, identification, or detection of the biosensors and/or which increases the solubility of the biosensors. In certain embodiments, the chemical handle may be represented by the formula:
X₍ₐ₎-R_{(b)}-Y_{(c)}
wherein:
X is selected from the group consisting of disulfide, sulfide, diselenide, selenide, thiol, isonitrile, selenol, a trivalent phosphorus compound, isothiocyanate, isocyanate, xanthanate, thiocarbamate, a phosphine, an amine, thio acid, dithio acid, monohalosilane, dihalosilane, trihalosilane, trialkoxysilane, dialkoxysilane, monoalkoxysilane, olefin, phosphate, carboxylic acid, alkylphosphoric acid, hydroxamic acid, diacylperoxides, peroxides, azo, alkynes, cyano, isonitrile, hydroxyl, carboxyl, vinyl, sulfonyl, phosphoryl, silicon hydride, and amino;
R is a linear or branched hydrocarbon chain from about 1 to about 400 carbons long optionally including in the chain —O—, —CONH—, —CONHCO—, —NH—, —CSNH—, —CO—, —CS—, —S—, —SO—, —(OCH₂CH₂)ₙ—, or —(CF₂)ₙ—;
Y is selected from the group consisting of hydroxyl, carboxyl, amino, aldehyde, carbonyl, methyl, methylene, alkene, alkyne, carbonate, aryliodide, vinyl, maleimide, N-hydroxysuccinimide, nitrilotriacetic acid, haloacetyl, bromoacetyl, iodoacetyl, activated carboxyl, hydrazide, epoxy, aziridine, sulfonylchloride, trifluoromethyldiaziridine, pyridyldisulfide, N-acyl-imidazole, imidazolecarbamate, vinylsulfone, succinimidylcarbonate, arylazide, anhydride, diazoacetate, benzophenone, isothiocyanate, isocyanate, imidoester, fluorobenzene, biotin, —RSR, —PO₄⁻³, —OSO₃⁻², —SO₃⁻, —COO⁻, —SOO⁻, —CONR₂, and —CN;
(a) is an integer from about 0 to about 4;
(b) is 0 or 1;
(c) is an integer greater than 0;
n is an integer from about 1 to about 22; and
R is H, alkyl, or aryl.

In other embodiments, the chemical handle may be selected from the group consisting of glutathione S-transferase (GST), protein A, protein G, calmodulin-binding peptide, thioredoxin, maltose binding protein, HA, myc, poly arginine, poly His, poly His-Asp, FLAG tag, a signal peptide, type III secretion system-targeting peptide, transcytosis domain, and nuclear localization signal.

In certain embodiments, the biosensor may be immobilized onto a substrate surface, including, for example, substrates such as silicon, silica, quartz, glass, controlled pore glass, carbon, alumina, titania, tantalum oxide, germanium, silicon nitride, zeolites, gallium arsenide, gold, platinum, aluminum, copper, titanium, alloys, polystyrene, poly(tetra)fluoroethylene (PTFE), polyvinylidenedifluoride, polycarbonate, polymethylmethacrylate, polyvinylethylene, polyethyleneimine, poly(etherether)ketone, polyoxymethylene (POM), polyvinylphenol, polylactides, polymethacrylimide (PMI), polyalkenesulfone (PAS), polypropylethylene, polyethylene, polyhydroxyethylmethacrylate (HEMA), polydimethylsiloxane, polyacrylamide, polyimide, and block-copolymers. Such substrates may be in the form of beads, chips, plates, slides, strips, sheets, films, blocks, plugs, medical devices, surgical instruments, diagnostic instruments, drug delivery devices, prosthetic implants, and other structures.

In another embodiment, the application provides a composition comprising two or more biosensors. The composition may comprise a pharmaceutically acceptable carrier. The biosensors of the composition may be specific for different target molecules and may be associated with the same or different reporter molecules.

In another embodiment, two or more biosensors may be immobilized onto a substrate at spatially addressable locations. The biosensors may be specific for different target molecules and may be associated with the same or different reporter molecules.

In another aspect, the application provides a method for detecting at least one target molecule comprising providing at least one biosensor comprising a selectivity component and a reporter molecule and detecting the signal of the reporter molecule, wherein interaction of the biosensor with the target molecule produces a detectable change in the signal of the reporter molecule. In various other aspects, the biosensors of the invention may be used for the detection of environmental pollutants, hazardous substances, food contaminants, and biological and/or chemical warfare agents.

In various embodiments, the biosensors of the invention may be used to detect target molecules, including, for example, cells, microorganisms (bacteria, fungi and viruses), polypeptides, nucleic acids, hormones, cytokines, drug molecules, carbohydrates, pesticides, dyes, amino acids, small organic molecules and small inorganic molecules. Biosensors may be used for the detection of target molecules both in vivo and in vitro. In certain embodiments, the biosensor may be injected or implanted into a patient and the signal of the reporter molecule is detected externally. In one exemplary embodiment, the biosensors of the application may be used for the detection of intracellular targets. In another exemplary embodiment, the biosensors of the application may be attached to a fiberoptic probe to facilitate position of the biosensor within a sample and readout from the biosensor through the optical fiber.

### DESCRIPTION

1. General

To provide an overall understanding, certain illustrative embodiments will now be described; however, it will be understood by one of ordinary skill in the art that the systems and methods described herein can be adapted and modified to provide systems and methods for other suitable applications and that other additions and modifications can be made without departing from the scope of the systems and methods described herein.

Unless otherwise specified, the illustrated embodiments can be understood as providing exemplary features of varying detail of certain embodiments, and therefore unless otherwise specified, features, components, modules, and/or aspects of the illustrations can be combined, separated, interchanged, and/or rearranged without departing from the disclosed systems or methods.

The present invention provides biosensors, compositions comprising biosensors, and methods of using biosensors. As described herein, the biosensors comprise a selectivity component capable of interacting with a target molecule and a reporter molecule that produces a detectable change in signal upon interaction of the selectivity component with a target molecule. The selectivity component may be a polypeptide (including antibodies and non-antibody receptor molecules, and fragments and variants thereof), polynucleotides (including aptamers), template imprinted materials, and organic and inorganic binding elements. The reporter molecule may be sensitive to changes in the environment, including, for example, pH sensitive molecules, polarity sensitive molecules, restriction sensitive molecules, or mobility sensitive molecules. The biosensor may optionally comprise a chemical handle suitable to facilitate isolation, immobilization, identification, or detection of the biosensors and/or which increases the solubility of the biosensors.

The biosensors described herein are useful for both in vivo and in vitro applications. In various embodiments, the biosensors may be used for detecting one or more target molecules, detecting environmental pollutants, detecting chemical or biological warfare agents, detecting food contaminants, and detecting hazardous substances. In an exemplary embodiment, the biosensors may be used for intracellular monitoring of one or more target molecules. The biosensors of the invention may be immobilized onto to a substrate surface, including, for example, a bead, chip, plate, slide, strip, sheet, film, block, plug, medical device, surgical instrument, diagnostic instrument, drug delivery device, prosthetic implant or other structure. Two or more biosensors may be used to form a panel or array of biosensors for monitoring multiple target molecules. In an exemplary embodiment, an array of 2, 10, 50, 100, 1000 or more biosensors are immobilized at spatially addressable locations on a substrate suitable for in vitro or in vivo applications.

2. Definitions

For convenience, certain terms employed in the specification, examples, and appended claims are collected here. Unless defined otherwise, all technical and scientific terms used herein have the same meaning as commonly understood by one of ordinary skill in the art to which this invention belongs.

The articles “a” and “an” are used herein to refer to one or to more than one (i.e., to at least one) of the grammatical object of the article. By way of example, “an element” means one element or more than one element.

The term “amino acid” is intended to embrace all molecules, whether natural or synthetic, which include both an amino functionality and an acid functionality and capable of being included in a polymer of naturally-occurring amino acids. Exemplary amino acids include naturally-occurring amino acids; analogs, derivatives and congeners thereof; amino acid analogs having variant side chains; and all stereoisomers of any of any of the foregoing.

The term “antibody” refers to an immunoglobulin, derivatives thereof which maintain specific binding ability, and proteins having a binding domain which is homologous or largely homologous to an immunoglobulin binding domain. These proteins may be derived from natural sources, or partly or wholly synthetically produced. An antibody may be monoclonal or polyclonal. The antibody may be a member of any immunoglobulin class, including any of the human classes: IgG, IgM, IgA, IgD, and IgE. In exemplary embodiments, antibodies used with the methods and compositions described herein are derivatives of the IgG class.

The term “antibody fragment” refers to any derivative of an antibody which is less than full-length. In exemplary embodiments, the antibody fragment retains at least a significant portion of the full-length antibody's specific binding ability. Examples of antibody fragments include, but are not limited to, Fab, Fab′, F(ab′)₂, scFv, Fv, dsFv diabody, and Fd fragments. The antibody fragment may be produced by any means. For instance, the antibody fragment may be enzymatically or chemically produced by fragmentation of an intact antibody, it may be recombinantly produced from a gene encoding the partial antibody sequence, or it may be wholly or partially synthetically produced. The antibody fragment may optionally be a single chain antibody fragment. Alternatively, the fragment may comprise multiple chains which are linked together, for instance, by disulfide linkages. The fragment may also optionally be a multimolecular complex. A functional antibody fragment will typically comprise at least about 50 amino acids and more typically will comprise at least about 200 amino acids.

The term “aptamer” refers to a nucleic acid molecule that may selectively interact with a non-oligonucleotide molecule or group of molecules. In various embodiments, aptamers may include single-stranded, partially single-stranded, partially double-stranded or double-stranded nucleic acid sequences; sequences comprising nucleotides, ribonucleotides, deoxyribonucleotides, nucleotide analogs, modified nucleotides and nucleotides comprising backbone modifications, branchpoints and normucleotide residues, groups or bridges; synthetic RNA, DNA and chimeric nucleotides, hybrids, duplexes, heteroduplexes; and any ribonucleotide, deoxyribonucleotide or chimeric counterpart thereof and/or corresponding complementary sequence. In certain embodiments, aptamers may include promoter or primer-annealing sequences that may be used to amplify, transcribe or replicate all or part of the aptamer molecule or sequence. As used herein, aptamers may also be referred to as nucleic acid ligands.

As used herein, the term “array” refers to a set of selectivity components immobilized onto one or more substrates so that each selectivity component is at a known location. In an exemplary embodiment, a set of selectivity components is immobilized onto a surface in a spatially addressable manner so that each individual selectivity component is located at different and identifiable location on the substrate.

The term “camelized antibody” refers to an antibody or variant thereof that has been modified to increase its solubility and/or reduce aggregation or precipitation. For example, camelids produce heavy-chain antibodies consisting only of a pair of heavy chains wherein the antigen binding site comprises the N-terminal variable region or VHH (variable domain of a heavy chain antibody). The VHH domain comprises an increased number of hydrophilic amino acid residues that enhance the solubility of a VHH domain as compared to a V_{H} region from non-camelid antibodies. Camelization of an antibody or variant thereof involves replacing one or more amino acid residues of a non-camelid antibody with corresponding amino residues from a camelid antibody.

The term “chemical handle” refers to a component that may be attached to a biosensor as described herein so as to facilitate its isolation, immobilization, identification, or detection and/or which increases its solubility. Suitable chemical handles include, for example, a polypeptide, a polynucleotide, a carbohydrate, a polymer, or a chemical moiety and combinations or variants thereof.

The term “conserved residue” refers to an amino acid that is a member of a group of amino acids having certain common properties. The term “conservative amino acid substitution” refers to the substitution (conceptually or otherwise) of an amino acid from one such group with a different amino acid from the same group. A functional way to define common properties between individual amino acids is to analyze the normalized frequencies of amino acid changes between corresponding proteins of homologous organisms (Schulz, G. E. and R. H. Schirmer., Principles of Protein Structure, Springer-Verlag). According to such analyses, groups of amino acids may be defined where amino acids within a group exchange preferentially with each other, and therefore resemble each other most in their impact on the overall protein structure (Schulz, G. E. and R. H. Schirmer, Principles of Protein Structure, Springer-Verlag). One example of a set of amino acid groups defined in this manner include: (i) a charged group, consisting of Glu and Asp, Lys, Arg and His, (ii) a positively-charged group, consisting of Lys, Arg and His, (iii) a negatively-charged group, consisting of Glu and Asp, (iv) an aromatic group, consisting of Phe, Tyr and Trp, (v) a nitrogen ring group, consisting of His and Trp, (vi) a large aliphatic nonpolar group, consisting of Val, Leu and Ile, (vii) a slightly-polar group, consisting of Met and Cys, (viii) a small-residue group, consisting of Ser, Thr, Asp, Asn, Gly, Ala, Glu, Gln and Pro, (ix) an aliphatic group consisting of Val, Leu, Ile, Met and Cys, and (x) a small hydroxyl group consisting of Ser and Thr.

The term “diabodies” refers to dimeric scFvs. The components of diabodies typically have shorter peptide linkers than most scFvs and they show a preference for associating as dimers. The term diabody is intended to encompass both bivalent (i.e., a dimer of two scFvs having the same specificity) and bispecific (i.e., a dimer of two scFvs having different specificities) molecules. Methods for preparing diabodies are known in the art. See, for example, EP 404097 and WO 93/11161.

As used herein, the term “epitope” refers to a physical structure on a molecule that interacts with a selectivity component. In exemplary embodiments, epitope refers to a desired region on a target molecule that specifically interacts with a selectivity component.

The term “Fab” refers to an antibody fragment that is essentially equivalent to that obtained by digestion of immunoglobulin (typically IgG) with the enzyme papain. The heavy chain segment of the Fab fragment is the Fd piece. Such fragments may be enzymatically or chemically produced by fragmentation of an intact antibody, recombinantly produced from a gene encoding the partial antibody sequence, or it may be wholly or partially synthetically produced. Methods for preparing Fab fragments are known in the art. See, for example, Tijssen, Practice and Theory of Enzyme Immunoassays (Elsevieer, Amsterdam, 1985).

The term “Fab′” refers to an antibody fragment that is essentially equivalent to that obtained by reduction of the disulfide bridge or bridges joining the two heavy chain pieces in the F(ab′)₂ fragment. Such fragments may be enzymatically or chemically produced by fragmentation of an intact antibody, recombinantly produced from a gene encoding the partial antibody sequence, or it may be wholly or partially synthetically produced.

The term “F(ab′)₂” refers to an antibody fragment that is essentially equivalent to a fragment obtained by digestion of an immunoglobulin (typically IgG) with the enzyme pepsin at pH 4.0-4.5. Such fragments may be enzymatically or chemically produced by fragmentation of an intact antibody, recombinantly produced from a gene encoding the partial antibody sequence, or it may be wholly or partially synthetically produced.

The term “Fv” refers to an antibody fragment that consists of one V_{H} and one V_{L} domain held together by noncovalent interactions. The term “dsFv” is used herein to refer to an Fv with an engineered intermolecular disulfide bond to stabilize the V_{H}-V_{L} pair. Methods for preparing Fv fragments are known in the art. See, for example, Moore et al., U.S. Pat. No. 4,462,334; Hochman et al., Biochemistry 12: 1130 (1973); Sharon et al., Biochemistry 15: 1591 (1976); and Ehrilch et al., U.S. Pat. No. 4,355,023.

The term “immunogen” traditionally refers to compounds that are used to elicit an immune response in an animal, and is used as such herein. However, many techniques used to produce a desired selectivity component, such as the phage display and aptamer methods described below, do not rely wholly, or even in part, on animal immunizations. Nevertheless, these methods use compounds containing an “epitope,” as defined above, to select for and clonally expand a population of selectivity components specific to the “epitope.” These in vitro methods mimic the selection and clonal expansion of immune cells in vivo, and, therefore, the compounds containing the “epitope” that is used to clonally expand a desired population of phage, aptamers and the like in vitro are embraced within the definition of “immunogens.”

Similarly, the terms “hapten” and “carrier” have specific meaning in relation to the immunization of animals, that is, a “hapten” is a small molecule that contains an epitope, but is incapable as serving as an immunogen, alone. Therefore, to elicit an immune response to the hapten, the hapten is conjugated with a larger carrier, such as bovine serum albumin or keyhole limpet hemocyanin, to produce an immunogen. A preferred immune response would recognize the epitope on the hapten, but not on the carrier. As used herein in connection with the immunization of animals, the terms “hapten” and “carrier” take on their classical definition. However, in the in vitro methods described herein for preparing the desired binding reagents, traditional “haptens” and “carriers” typically have their counterpart in epitope-containing compounds affixed to suitable substrates or surfaces, such as beads and tissue culture plates.

The term “mammal” is known in the art, and exemplary mammals include humans, primates, bovines, porcines, canines, felines, and rodents (e.g., mice and rats).

The term “microenvironment” refers to localized conditions within a larger area. For example, association of two molecules within a solution may alter the local conditions surrounding the associating molecules without affecting the overall conditions within the solution.

The term “nucleic acid” refers to a polymeric form of nucleotides, either ribonucleotides or deoxynucleotides or a modified form of either type of nucleotide. The terms should also be understood to include, as equivalents, analogs of either RNA or DNA made from nucleotide analogs, and, as applicable to the embodiment being described, single-stranded (such as sense or antisense) and double-stranded polynucleotides.

The term “polypeptide”, and the terms “protein” and “peptide” which are used interchangeably herein, refers to a polymer of amino acids.

The terms “polypeptide fragment” or “fragment”, when used in regards to a reference polypeptide, refers to a polypeptide in which amino acid residues are deleted as compared to the reference polypeptide itself, but where the remaining amino acid sequence is usually identical to the corresponding positions in the reference polypeptide. Such deletions may occur at the amino-terminus or carboxy-terminus of the reference polypeptide, or alternatively both. Fragments typically are at least 5, 10, 20, 50, 100, 500 or more amino acids long. A fragment can retain one or more of the biological activities of the reference polypeptide.

As used herein, the term “reporter molecule” refers to a molecule suitable for detection, such as, for example, spectroscopic detection. Examples of reporter molecules include, but are not limited to, the following: radioisotopes, fluorescent labels, heavy atoms, enzymatic labels, chemiluminescent groups, biotinyl groups, and predetermined polypeptide epitopes recognized by a secondary reporter (e.g., leucine zipper pair sequences, binding sites for secondary antibodies, metal binding domains, epitope tags). Examples and use of such reporter molecules are described in more detail below. In some embodiments, reporter molecules are attached by spacer arms of various lengths to reduce potential steric hindrance. Reporter molecules may be incorporated into or attached (including covalent and non-covalent attachment) to a molecule, such as a selectivity component. Various methods of labeling polypeptides are known in the art and may be used.

The terms “single-chain Fvs” and “scFvs” refers to recombinant antibody fragments consisting of only the variable light chain (V_{L}) and variable heavy chain (V_{H}) covalently connected to one another by a polypeptide linker. Either V_{L} or V_{H} may be the NH₂-terminal domain. The polypeptide linker may be of variable length and composition so long as the two variable domains are bridged without serious steric interference. In exemplary embodiments, the linkers are comprised primarily of stretches of glycine and serine residues with some glutamic acid or lysine residues interspersed for solubility. Methods for preparing scFvs are known in the art. See, for example, PCT/US/87/02208 and U.S. Pat. No. 4,704,692.

The term “single domain antibody” or “Fd” refers to an antibody fragment comprising a V_{H} domain that interacts with a given antigen. An Fd does not contain a V_{L} domain, but may contain other antigen binding domains known to exist in antibodies, for example, the kappa and lambda domains. Methods for preparing Fds are known in the art. See, for example, Ward et al., Nature 341:644-646 (1989) and EP 0368684 A1.

The term “single chain antibody” refers to an antibody fragment that comprises variable regions of the light and heavy chains joined by a flexible linker moiety. Methods for preparing single chain antibodies are known in the art. See, for example, U.S. Pat. No. 4,946,778 to Ladner et al.

As used herein, the term “selectivity component” refers to a molecule capable of interacting with a target molecule. Selectivity components having limited cross-reactivity are generally preferred. In certain embodiments, suitable selectivity components include, for example, antibodies, monoclonal antibodies, or derivatives or analogs thereof, including without limitation: Fv fragments, single chain Fv (scFv) fragments, Fab′ fragments, F(ab′)2 fragments, single domain antibodies, camelized antibodies and antibody fragments, humanized antibodies and antibody fragments, and multivalent versions of the foregoing; multivalent binding reagents including without limitation: monospecific or bispecific antibodies, such as disulfide stabilized Fv fragments, scFv tandems ((scFv)₂ fragments), diabodies, tribodies or tetrabodies, which typically are covalently linked or otherwise stabilized (i.e., leucine zipper or helix stabilized) scFv fragments; and other binding reagents including, for example, aptamers, template imprinted materials (such as those of U.S. Pat. No. 6,131,580), and organic or inorganic binding elements. In exemplary embodiments, a selectivity component specifically interacts with a single epitope. In other embodiments, a selectivity component may interact with several structurally related epitopes.

As used herein, the term “sensor dye” refers to a reporter molecule that exhibits an increase, decrease or modification of signal in response to a change in the environment. In exemplary embodiments, the sensor dye is a fluorescent molecule that is response to changes in pH, polarity, viscosity, and/or mobility.

The term “triabody” refers to trivalent constructs comprising 3 scFv's, and thus comprising 3 variable domains (see, e.g., Iliades et al., FEBS Lett. 409(3):437-41 (1997)). Triabodies is meant to include molecules that comprise 3 variable domains having the same specificity, or 3 variable domains wherein two or more of the variable domains have different specificities.

The term “tetrabody” refers to engineered antibody constructs comprising 4 variable domains (see, e.g., Pack et al., J Mol. Biol. 246(1): 28-34 (1995) and Coloma & Morrison, Nat Biotechnol. 15(2): 159-63 (1997)). Tetrabodies is meant to include molecules that comprise 4 variable domains having the same specificity, or 4 variable domains wherein two or more of the variable domains have different specificities.

The term “V_{H}” refers to a heavy chain variable region of an antibody.

The term “V_{L}” refers to a light chain variable region of an antibody.

3. Selectivity Component

The selectivity component may be any molecule which is capable of selectively interacting with a desired target, including, for example, cells, microorganisms (such as bacteria, fungi and viruses), polypeptides, nucleic acids (such as oligonucleotides, cDNA molecules or genomic DNA fragments), hormones, cytokines, drug molecules, carbohydrates, pesticides, dyes, amino acids, or small organic or inorganic molecules. Exemplary target molecules include, for example, molecules involved in tissue differentiation and/or growth, cellular communication, cell division, cell motility, and other cellular functions that take place within or between cells, including regulatory molecules such as growth factors, cytokines, morphogenetic factors, neurotransmitters, and the like. In certain embodiments, target molecules may be bone morphogenic protein, insulin-like growth factor (IGF), and/or members of the hedgehog and Wnt polypeptide families. Exemplary selectivity components include, for example, antibodies, antibody fragments, non-antibody receptor molecules, aptamers, template imprinted materials, and organic or inorganic binding elements. Selectivity components having limited cross-reactivity are generally preferred.

In certain embodiments, the selectivity component may be an antibody or an antibody fragment. For example, selectivity components may be monoclonal antibodies, or derivatives or analogs thereof, including without limitation: Fv fragments, single chain Fv (scFv) fragments, Fab′ fragments, F(ab′)2 fragments, single domain antibodies, camelized antibodies and antibody fragments, humanized antibodies and antibody fragments, and multivalent versions of the foregoing; multivalent selectivity components including without limitation: monospecific or bispecific antibodies, such as disulfide stabilized Fv fragments, scFv tandems ((scFv)₂ fragments), diabodies, tribodies or tetrabodies, which typically are covalently linked or otherwise stabilized (i.e., leucine zipper or helix stabilized) scFv fragments; receptor molecules which naturally interact with a desired target molecule.

In one embodiment, the selectivity component may be an antibody. Preparation of antibodies may be accomplished by any number of well-known methods for generating monoclonal antibodies. These methods typically include the step of immunization of animals, typically mice, with a desired immunogen (e.g., a desired target molecule or fragment thereof). Once the mice have been immunized, and preferably boosted one or more times with the desired immunogen(s), monoclonal antibody-producing hybridomas may be prepared and screened according to well known methods (see, for example, Kuby, Janis, IMMUNOLOGY, Third Edition, pp. 131-139, W.H. Freeman & Co. (1997), for a general overview of monoclonal antibody production, that portion of which is incorporated herein by reference).

Over the past several decades, antibody production has become extremely robust. In vitro methods that combine antibody recognition and phage display techniques allow one to amplify and select antibodies with very specific binding capabilities. See, for example, Holt, L. J. et al., “The Use of Recombinant Antibodies in Proteomics,” Current Opinion in Biotechnology 2000, 11:445-449, incorporated herein by reference. These methods typically are much less cumbersome than preparation of hybridomas by traditional monoclonal antibody preparation methods. Binding epitopes may range in size from small organic compounds such as bromo uridine and phosphotyrosine to oligopeptides on the order of 7-9 amino acids in length.

In another embodiment, the selectivity component may be an antibody fragment. Preparation of antibody fragments may be accomplished by any number of well-known methods. In one embodiment, phage display technology may be used to generate antibody fragment selectivity components that are specific for a desired target molecule, including, for example, Fab fragments, Fv's with an engineered intermolecular disulfide bond to stabilize the V_{H}-V_{L} pair, scFvs, or diabody fragments. As an example, production of scFv antibody fragments using phage display is described below.

For phage display, an immune response to a selected immunogen is elicited in an animal (such as a mouse, rabbit, goat or other animal) and the response is boosted to expand the immunogen-specific B-cell population. Messenger RNA is isolated from those B-cells, or optionally a monoclonal or polyclonal hybridoma population. The mRNA is reverse-transcribed by known methods using either a poly-A primer or murine immunoglobulin-specific primer(s), typically specific to sequences adjacent to the desired V_{H} and V_{L} chains, to yield cDNA. The desired V_{H} and V_{L} chains are amplified by polymerase chain reaction (PCR) typically using V_{H} and V_{L} specific primer sets, and are ligated together, separated by a linker. V_{H} and V_{L} specific primer sets are commercially available, for instance from Stratagene, Inc. of La Jolla, Calif. Assembled V_{H}-linker-V_{L} product (encoding an scFv fragment) is selected for and amplified by PCR. Restriction sites are introduced into the ends of the V_{H}-linker-V_{L} product by PCR with primers including restriction sites and the scFv fragment is inserted into a suitable expression vector (typically a plasmid) for phage display. Other fragments, such as an Fab′ fragment, may be cloned into phage display vectors for surface expression on phage particles. The phage may be any phage, such as lambda, but typically is a filamentous phage, such as fd and M13, typically M13.

In phage display vectors, the V_{H}-linker-V_{L} sequence is cloned into a phage surface protein (for M13, the surface proteins g3p (pIII) or g8p, most typically g3p). Phage display systems also include phagemid systems, which are based on a phagemid plasmid vector containing the phage surface protein genes (for example, g3p and g8p of M13) and the phage origin of replication. To produce phage particles, cells containing the phagemid are rescued with helper phage providing the remaining proteins needed for the generation of phage. Only the phagemid vector is packaged in the resulting phage particles because replication of the phagemid is grossly favored over replication of the helper phage DNA. Phagemid packaging systems for production of antibodies are commercially available. One example of a commercially available phagemid packaging system that also permits production of soluble ScFv fragments in bacteria cells is the Recombinant Phage Antibody System (RPAS), commercially available from Amersham Pharmacia Biotech, Inc. of Piscataway, N.J. and the pSKAN Phagemid Display System, commercially available from MoBiTec, LLC of Marco Island, Florida. Phage display systems, their construction and screening methods are described in detail in, among others, U.S. Pat. Nos. 5,702,892, 5,750,373, 5,821,047 and 6,127,132, each of which are incorporated herein by reference in their entirety.

Typically, once phage are produced that display a desired antibody fragment, epitope-specific phage are selected by their affinity for the desired immunogen and, optionally, their lack of affinity to compounds containing certain other structural features. A variety of methods may be used for physically separating immunogen-binding phage from non-binding phage. Typically the immunogen is fixed to a surface and the phage are contacted with the surface. Non-binding phage are washed away while binding phage remain bound. Bound phage are later eluted and are used to re-infect cells to amplify the selected species. A number of rounds of affinity selection typically are used, often increasingly higher stringency washes, to amplify immunogen-binding phage of increasing affinity. Negative selection techniques also may be used to select for lack of binding to a desired target. In that case, un-bound (washed) phage are amplified.

Although it is preferred to use spleen cells and/or B-lymphocytes from animals pre-immunized with a desired immunogen as a source of cDNA from which the sequences of the V_{H} and V_{L} chains are amplified by RT-PCR, naive (un-immunized with the target immunogen) splenocytes and/or B-cells may be used as a source of cDNA to produce a polyclonal set of V_{H} and V_{L} chains that are selected in vitro by affinity, typically by the above-described phage display (phagemid) method. When naive B-cells are used, during affinity selection, the washing of the first selection step typically is of very low stringency so as to avoid loss of any single clone that may be present in very low copy number in the polyclonal phage library. By this naive method, B-cells may be obtained from any polyclonal source. B-cell or splenocyte cDNA libraries also are a source of cDNA from which the V_{H} and V_{L} chains may be amplified. For example, suitable murine and human B-cell, lymphocyte and splenocyte cDNA libraries are commercially available from Stratagene, Inc. and from Clontech Laboratories, Inc. of Palo Alto, Calif. Phagemid antibody libraries and related screening services are provided commercially by Cambridge Antibody Technology of the U.K. or MorphoSys USA, Inc., of Charlotte, N.C.

The selectivity components do not have to originate from biological sources, such as from naive or immunized immune cells of animals or humans. The selectivity components may be screened from a combinatorial library of synthetic peptides. One such method is described in U.S. Pat. No. 5,948,635, incorporated herein by reference, which described the production of phagemid libraries having random amino acid insertions in the pIII gene of M13. These phage may be clonally amplified by affinity selection as described above.

Panning in a culture dish or flask is one way to physically separate binding phage from non-binding phage. Panning may be carried out in 96 well plates in which desired immunogen structures have been immobilized. Functionalized 96 well plates, typically used as ELISA plates, may be purchased from Pierce of Rockwell, Illinois. Polypeptides immunogens may be synthesized directly on NH₂ or COOH functionalized plates in an N-terminal to C-terminal direction. Other affinity methods for isolating phage having a desired specificity include affixing the immunogen to beads. The beads may be placed in a column and phage may be bound to the column, washed and eluted according to standard procedures. Alternatively, the beads may be magnetic so as to permit magnetic separation of the binding particles from the non-binding particles. The immunogen also may be affixed to a porous membrane or matrix, permitting easy washing and elution of the binding phage.

In certain embodiments, it may be desirable to increase the specificity of the selectivity component for a given target molecule using a negative selection step in the affinity selection process. For example, selectivity component displaying phage may be contacted with a surface funtionalized with immunogens distinct from the target molecule. Phage are washed from the surface and non-binding phage are grown to clonally expand the population of non-binding phage thereby de-selecting phage that are not specific for the desired target molecule. In certain embodiments, random synthetic peptides may be used in the negative selection step. In other embodiments, one or more immunogens having structural similarity to the target molecule may be used in the negative selection step. For example, for a target molecule comprising a polypeptide, structurally similar immunogens may be polypeptides having conservative amino acid substitutions, including but not limited to the conservative substitution groups such as: (i) a charged group, consisting of Glu and Asp, Lys, Arg and His, (ii) a positively-charged group, consisting of Lys, Arg and His, (iii) a negatively-charged group, consisting of Glu and Asp, (iv) an aromatic group, consisting of Phe, Tyr and Trp, (v) a nitrogen ring group, consisting of His and Trp, (vi) a large aliphatic nonpolar group, consisting of Val, Leu and Ile, (vii) a slightly-polar group, consisting of Met and Cys, (viii) a small-residue group, consisting of Ser, Thr, Asp, Asn, Gly, Ala, Glu, Gln and Pro, (ix) an aliphatic group consisting of Val, Leu, Ile, Met and Cys, and (x) a small hydroxyl group consisting of Ser and Thr. Conservative substitutions also may be determined by one or more methods, such as those used by the BLAST (Basic Local Alignment Search Tool) algorithm, such as a BLOSUM Substitution Scoring Matrix, such as the BLOSUM 62 matrix, and the like. A functional way to define common properties between individual amino acids is to analyze the normalized frequencies of amino acid changes between corresponding proteins of homologous organisms (Schulz, G. E. and R. H. Schirmer, Principles of Protein Structure, Springer-Verlag).

Screening of selectivity components will best be accomplished by high throughput parallel selection, as described in Holt et al. Alternatively, high throughput parallel selection may be conducted by commercial entities, such as by Cambridge Antibody Technologies or MorphoSys USA, Inc.

Alternatively, selection of a desired selectivity component-displaying phage may be carried out using the following method:

Step 1: Affinity purify phage under low stringency conditions for their ability to bind to an immunogen fixed to a solid support (for instance, beads in a column).

Step 2: Elute the bound phage and grow the eluted phage. Steps 1 and 2 may be repeated with more stringent washes in Step 1.

Step 3: Absorb the phage under moderate stringency with a given protein mixture digested with a proteolytic agent of interest. Wash away the unbound phage with a moderately stringent wash and grow the washed phage. Step 3 may be repeated with less stringent washes.

Step 4: Affinity purify phage under high stringency for their ability to bind to the immunogen fixed to a solid support. Elute the bound phage and grow the eluted phage.

Step 5: Plate the phage to select single plaques. Independently grow phage selected from each plaque and confirm the specificity to the desired immunogen.

This is a general guideline for the clonal expansion of immunogen-specific selectivity components. Additional steps of varying stringency may be added at any stage to optimize the selection process, or steps may be omitted or re-ordered. One or more steps may be added where the phage population is selected for its inability to bind to other immunogens by absorption of the phage population with those other immunogens and amplification of the unbound phage population. That step may be performed at any stage, but typically would be performed after step 4.

In certain embodiments, it may be desirable to mutate the binding region of the selectivity component and select for selectivity components with superior binding characteristics as compared to the un-mutated selectivity component. This may be accomplished by any standard mutagenesis technique, such as by PCR with Taq polymerase under conditions that cause errors. In such a case, the PCR primers could be used to amplify scFv-encoding sequences of phagemid plasmids under conditions that would cause mutations. The PCR product may then be cloned into a phagemid vector and screened for the desired specificity, as described above.

In other embodiments, the selectivity components may be modified to make them more resistant to cleavage by proteases. For example, the stability of the selectivity components of the present invention that comprise polypeptides may be increased by substituting one or more of the naturally occurring amino acids in the (L) configuration with D-amino acids. In various embodiments, at least 1%, 5%, 10%, 20%, 50%, 80%, 90% or 100% of the amino acid residues of the selectivity components may be of the D configuration. The switch from L to D amino acids neutralizes the digestion capabilities of many of the ubiquitous peptidases found in the digestive tract. Alternatively, enhanced stability of the selectivity components of the invention may be achieved by the introduction of modifications of the traditional peptide linkages. For example, the introduction of a cyclic ring within the polypeptide backbone may confer enhanced stability in order to circumvent the effect of many proteolytic enzymes known to digest polypeptides in the stomach or other digestive organs and in serum. In still other embodiments, enhanced stability of the selectivity components may be achieved by intercalating one or more dextrorotatory amino acids (such as, dextrorotatory phenylalanine or dextrorotatory tryptophan) between the amino acids of the selectivity component. In exemplary embodiments, such modifications increase the protease resistance of the selectivity components without affecting their activity or specificity of interaction with a desired target molecule.

In certain embodiments, the antibodies or variants thereof, may be modified to make them less immunogenic when administered to a subject. For example, if the subject is human, the antibody may be “humanized”; where the complimentarity determining region(s) of the hybridoma-derived antibody has been transplanted into a human monoclonal antibody, for example as described in Jones, P. et al. (1986), Nature 321, 522-525, Tempest et al. (1991) Biotechnology 9, 266-273, and U.S. Pat. No. 6,407,213. Also, transgenic mice, or other mammals, may be used to express humanized antibodies. Such humanization may be partial or complete.

In another embodiment, the selectivity component is an Fab fragment. Fab antibody fragments may be obtained by proteolysis of an immunoglobulin molecule using the protease papain. Papain digestion yields two identical antigen-binding fragments, termed “Fab fragments”, each with a single antigen-binding site, and a residual “Fc fragment”. In an exemplary embodiment, papain is first activated by reducing the sulfhydryl group in the active site with cysteine, mercaptoethanol or dithiothreitol. Heavy metals in the stock enzyme may be removed by chelation with EDTA (2 mM) to ensure maximum enzyme activity. Enzyme and substrate are normally mixed together in the ratio of 1: 100 by weight. After incubation, the reaction can be stopped by irreversible alkylation of the thiol group with iodoacetamide or simply by dialysis. The completeness of the digestion should be monitored by SDS-PAGE and the various fractions separated by protein A-Sepharose or ion exchange chromatography.

In still another embodiment, the selectivity component is an F(ab′)₂ fragment. F(ab′)₂ antibody fragments may be prepared from IgG molecules using limited proteolysis with the enzyme pepsin. Exemplary conditions for pepsin proteolysis are 100 times antibody excess w/w in acetate buffer at pH 4.5 and 37° C. Pepsin treatment of intact immunoglobulin molecules yields an F(ab′)₂ fragment that has two antigen-combining sites and is still capable of cross-linking antigen. Fab′ antibody fragments may be obtained by reducing F(ab′)₂ fragments using 2-mercaptoethylamine. The Fab′ fragments may be separated from unsplit F(ab′)₂ fragments and concentrated by application to a Sephadex G-25 column (Mᵣ=46,000-58,000).

In other embodiments, the selectivity component may be a non-antibody receptor molecule, including, for example, receptors which naturally recognize a desired target molecule, receptors which have been modified to increase their specificity of interaction with a target molecule, receptor molecules which have been modified to interact with a desired target molecule not naturally recognized by the receptor, and fragments of such receptor molecules (see, e.g., Skerra, J. Molecular Recognition 13: 167-187 (2000)).

In still other embodiments, the selectivity component may be an aptamer. Aptamers are oligonucleotides that are selected to bind specifically to a desired molecular structure. Aptamers typically are the products of an affinity selection process similar to the affinity selection of phage display (also known as in vitro molecular evolution). The process involves performing several tandem iterations of affinity separation, e.g., using a solid support to which the desired immunogen is bound, followed by polymerase chain reaction (PCR) to amplify nucleic acids that bound to the immunogens. Each round of affinity separation thus enriches the nucleic acid population for molecules that successfully bind the desired immunogen. In this manner, a random pool of nucleic acids may be “educated” to yield aptamers that specifically bind target molecules. Aptamers typically are RNA, but may be DNA or analogs or derivatives thereof, such as, without limitation, peptide nucleic acids (PNAs) and phosphorothioate nucleic acids.

In exemplary embodiments, nucleic acid ligands, or aptamers, may be prepared using the “SELEX” methodology which involves selection of nucleic acid ligands which interact with a target in a desirable manner combined with amplification of those selected nucleic acids. The SELEX process is described in U.S. Pat. Nos. 5,475,096 and 5,270,163 and PCT application No. WO 91/19813. These references, each specifically incorporated herein by reference, are collectively called the SELEX Patents.

The SELEX process provides a class of products which are nucleic acid molecules, each having a unique sequence, and each of which has the property of binding specifically to a desired target compound or molecule. In various embodiments, target molecules may be, for example, proteins, carbohydrates, peptidoglycans or small molecules. SELEX methodology can also be used to target biological structures, such as cell surfaces or viruses, through specific interaction with a molecule that is an integral part of that biological structure.

In its most basic form, the SELEX process may be defined by the following series of steps:
1) A candidate mixture of nucleic acids of differing sequence is prepared. The candidate mixture generally includes regions of fixed sequences (i.e., each of the members of the candidate mixture contains the same sequences in the same location) and regions of randomized sequences. The fixed sequence regions are selected either: (a) to assist in the amplification steps described below, (b) to mimic a sequence known to bind to the target, or (c) to enhance the concentration of a given structural arrangement of the nucleic acids in the candidate mixture. The randomized sequences can be totally randomized (i.e., the probability of finding a base at any position being one in four) or only partially randomized (e.g., the probability of finding a base at any location can be selected at any level between 0 and 100 percent).
2) The candidate mixture is contacted with the selected target under conditions favorable for binding between the target and members of the candidate mixture. Under these circumstances, the interaction between the target and the nucleic acids of the candidate mixture can be considered as forming nucleic acid-target pairs between the target and those nucleic acids having the strongest affinity for the target.
3) The nucleic acids with the highest affinity for the target are partitioned from those nucleic acids with lesser affinity to the target. Because only an extremely small number of sequences (and possibly only one molecule of nucleic acid) corresponding to the highest affinity nucleic acids exist in the candidate mixture, it is generally desirable to set the partitioning criteria so that a significant amount of the nucleic acids in the candidate mixture (approximately 5-50%) are retained during partitioning.
4) Those nucleic acids selected during partitioning as having the relatively higher affinity for the target are then amplified to create a new candidate mixture that is enriched in nucleic acids having a relatively higher affinity for the target.
5) By repeating the partitioning and amplifying steps above, the newly formed candidate mixture contains fewer and fewer unique sequences, and the average degree of affinity of the nucleic acids to the target will generally increase. The SELEX process ultimately may yield a candidate mixture containing one or a small number of unique nucleic acids representing those nucleic acids from the original candidate mixture having the highest affinity to the target molecule.

The basic SELEX method has been modified to achieve a number of specific objectives. For example, U.S. Pat. No. 5,707,796 describes the use of the SELEX process in conjunction with gel electrophoresis to select nucleic acid molecules with specific structural characteristics, such as bent DNA. U.S. Pat. No. 5,580,737 describes a method for identifying highly specific nucleic acid ligands able to discriminate between closely related molecules, termed Counter-SELEX. U.S. Pat. No. 5,567,588 describes a SELEX-based method which achieves highly efficient partitioning between oligonucleotides having high and low affinity for a target molecule. U.S. Pat. Nos. 5,496,938 and 5,683,867 describe methods for obtaining improved nucleic acid ligands after SELEX has been performed.

In certain embodiments, nucleic acid ligands as described herein may comprise modifications that increase their stability, including, for example, modifications that provide increased resistance to degradation by enzymes such as endonucleases and exonucleases, and/or modifications that enhance or mediate the delivery of the nucleic acid ligand (see, e.g., U.S. Pat. Nos. 5,660,985 and 5,637,459). Examples of such modifications include chemical substitutions at the ribose and/or phosphate and/or base positions. In various embodiments, modifications of the nucleic acid ligands may include, but are not limited to, those which provide other chemical groups that incorporate additional charge, polarizability, hydrophobicity, hydrogen bonding, electrostatic interaction, and fluxionality to the nucleic acid ligand bases or to the nucleic acid ligand as a whole. Such modifications include, but are not limited to, 2′-position sugar modifications, 5-position pyrimidine modifications, 8-position purine modifications, modifications at exocyclic amines, substitution of 4-thiouridine, substitution of 5-bromo or 5-iodo-uracil; backbone modifications, phosphorothioate or alkyl phosphate modifications, methylations, unusual base-pairing combinations such as the isobases isocytidine and isoguanidine and the like. Modifications may also include 3′ and 5′ modifications such as capping. In exemplary embodiments, the nucleic acid ligands are RNA molecules that are 2′-fluoro (2′-F) modified on the sugar moiety of pyrimidine residues.

In other embodiments, the selectivity components may be a template imprinted materials. Template imprinted materials are structures which have an outer sugar layer and an underlying plasma-deposited layer. The outer sugar layer contains indentations or imprints which are complementary in shape to a desired target molecule or template so as to allow specific interaction between the template imprinted structure and the target molecule to which it is complementary. Template imprinting can be utilized on the surface of a variety of structures, including, for example, medical prostheses (such as artificial heart valves, artificial limb joints, contact lenses and stents), microchips (preferably silicon-based microchips) and components of diagnostic equipment designed to detect specific microorganisms, such as viruses or bacteria. Template-imprinted materials are discussed in U.S. Pat. No. 6,131,580, which is hereby incorporated by reference in its entirety.

In certain embodiments, a selectivity component of the invention may contain a chemical handle which facilitates its isolation, immobilization, identification, or detection and/or which increases its solubility. In various embodiments, chemical handles may be a polypeptide, a polynucleotide, a carbohydrate, a polymer, or a chemical moiety and combinations or variants thereof. In certain embodiments, exemplary chemical handles, include, for example, glutathione S-transferase (GST), protein A, protein G, calmodulin-binding peptide, thioredoxin, maltose binding protein, HA, myc, poly arginine, poly His, poly His-Asp or FLAG tags. Additional exemplary chemical handles include polypeptides that alter protein localization in vivo, such as signal peptides, type III secretion system-targeting peptides, transcytosis domains, nuclear localization signals, etc. In various embodiments, a selectivity component of the invention may comprise one or more chemical handles, including multiple copies of the same chemical handle or two or more different chemical handles. It is also within the scope of the invention to include a linker (such as a polypeptide sequence or a chemical moiety) between a selectivity component of the invention and the chemical handle in order to facilitate construction of the molecule or to optimize its structural constraints. In another embodiment, the biosensor comprising a chemical handle may be constructed so as to contain protease cleavage sites between the chemical handle and the selectivity component of the invention in order to remove the chemical handle. Examples of suitable endoproteases for removal of a chemical handle, include, for example, Factor Xa and TEV proteases.

In another embodiment, a selectivity component of the invention may be modified so that its rate of traversing the cellular membrane is increased. For example, the selectivity component may be attached to a peptide which promotes “transcytosis,” e.g., uptake of a polypeptide by cells. The peptide may be a portion of the HIV transactivator (TAT) protein, such as the fragment corresponding to residues 37-62 or 48-60 of TAT, portions which have been observed to be rapidly taken up by a cell in vitro (Green and Loewenstein, (1989) Cell 55:1179-1188). Alternatively, the internalizing peptide may be derived from the Drosophila antennapedia protein, or homologs thereof. The 60 amino acid long homeodomain of the homeo-protein antennapedia has been demonstrated to translocate through biological membranes and can facilitate the translocation of heterologous polypeptides to which it is coupled. Thus, selectivity components may be fused to a peptide consisting of about amino acids 42-58 of Drosophila antennapedia or shorter fragments for transcytosis (Derossi et al. (1996) J Biol Chem 271:18188-18193; Derossi et al. (1994) J Biol Chem 269:10444-10450; and Perez et al. (1992) J Cell Sci 102:717-722). The transcytosis polypeptide may also be a non-naturally-occurring membrane-translocating sequence (MTS), such as the peptide sequences disclosed in U.S. Pat. No. 6,248,558.

In exemplary embodiments, the dissociation constant of the selectivity component for a target molecule is optimized to allow real time monitoring of the presence and/or concentration of the analyte in a given patient, sample, or environment.

The selectivity components (for example, phage, antibodies, antibody fragments, aptamers, etc.) may be affixed to a suitable substrate by a number of known methods. Typically the surface of the substrate is functionalized in some manner, so that a crosslinking compound or compounds may covalently link the selectivity component to the substrate. For example, a substrate functionalized with carboxyl groups may be linked to free amines in the selectivity components using EDC or by other common chemistries, such as by linking with N-hydroxysuccinimide. A variety of crosslinking chemistries are commercially available, for instance, from Pierce of Rockford, Ill.

For attachment of the sensor units to surfaces there are a number of traditional attachment technologies. For example, activated carboxyl groups on the substrate will link the sensor units to the substrate via —NH₂ groups on the selectivity component of the biosensor. The substrate of the array may be either organic or inorganic, biological or non-biological, or any combination of these materials. Numerous materials are suitable for use as a substrate for the sensor units of the invention. For instance, the substrate of the invention sensors can comprise a material selected from a group consisting of silicon, silica, quartz, glass, controlled pore glass, carbon, alumina, titania, tantalum oxide, germanium, silicon nitride, zeolites, and gallium arsenide. Many metals such as gold, platinum, aluminum, copper, titanium, and their alloys are also options for substrates of the array. In addition, many ceramics and polymers may also be used as substrates. Polymers which may be used as substrates include, but are not limited to, the following: polystyrene; poly(tetra)fluoroethylene (PTFE); polyvinylidenedifluoride; polycarbonate; polymethylmethacrylate; polyvinylethylene; polyethyleneimine; poly(etherether)ketone; polyoxymethylene (POM); polyvinylphenol; polylactides; polymethacrylimide (PMI); polyalkenesulfone (PAS); polypropylethylene, polyethylene; polyhydroxyethylmethacrylate (HEMA); polydimethylsiloxane; polyacrylamide; polyimide; and block-copolymers. Preferred substrates for the array include silicon, silica, glass, and polymers. The substrate on which the sensors reside may also be a combination of any of the aforementioned substrate materials.

A biosensor of the present invention may optionally further comprise a coating between the substrate and the bound biosensor molecule. This coating may either be formed on the substrate or applied to the substrate. The substrate can be modified with a coating by using thin-film technology based, for instance, on physical vapor deposition (PVD), plasma-enhanced chemical vapor deposition (PECVD), or thermal processing. Alternatively, plasma exposure can be used to directly activate or alter the substrate and create a coating. For instance, plasma etch procedures can be used to oxidize a polymeric surface (for example, polystyrene or polyethylene to expose polar functionalities such as hydroxyls, carboxylic acids, aldehydes and the like) which then acts as a coating.

The coating may also comprise a composition selected from the group consisting of silicon, silicon oxide, titania, tantalum oxide, silicon nitride, silicon hydride, indium tin oxide, magnesium oxide, alumina, glass, hydroxylated surfaces, and polymers.

The substrate surface shall comprise molecules of formula X₍ₐ₎-R_{(b)}-Y_{(c)}, wherein R is a spacer, X is a functional group that binds R to the surface, Y is a functional group for binding to the biosensor, (a) is an integer from 0 to about 4, (b) is either 0 or 1, and (c) is an integer not equal to 0. Note that when both (a) and (b) are zero, the substrate surface comprises functional groups Y as would be seen, for example, with polymeric substrates or coatings. When (a) and (b) are not equal to 0, then X₍ₐ₎-R_{(b)}-Y_{(c)} describes, for example, monolayers such as a self assembled monolayers that form on a metal surface. X₍ₐ₎-R_{(b)}-Y_{(c)} may also describe such compounds as 3-aminopropyltrimethoxysilane, wherein X is —Si(OMe)₃, R is —CH₂CH₂CH₂—, and Y is —NH₂. This compound is known to coat porous glass surfaces to form an aminopropyl derivative of the glass. Biochem. Biophys. Act., 1970, 212, 1; J. Chromatography, 1974, 97, 39.

Other definitions for R, X, and Y include the following. R optionally comprises a linear or branched hydrocarbon chain from about 1 to about 400 carbons long. The hydrocarbon chain may comprise an alkyl, aryl, alkenyl, alkynyl, cycloalkyl, alkaryl, aralkyl group, or any combination thereof. If (a) and (c) are both equal to one, then R is typically an alkyl chain from about 3 to about 30 carbons long. In a preferred embodiment, if (a) and (b) are both equal to one, then R is an alkyl, chain from about 8 to about 22 carbons long and is, optionally, a straight alkane. However, it is also contemplated that in an alternative embodiment, R may readily comprise a linear or branched hydrocarbon chain from about 2 to about 400 carbons long and be interrupted by at least one hetero atom. The interrupting hetero groups can include —O—, —CONH—, —CONHCO—, —NH—, —CSNH—, —CO—, —CS—, —S—, —SO—, —(OCH₂CH₂)ₙ— (where n=1-20), —(CF₂)ₙ— (where n=1-22), and the like. Alternatively, one or more of the hydrogen moieties of R can be substituted with deuterium. In alternative embodiments, R may be more than about 400 carbons long.

X may be chosen as any group which affords chemisorption or physisorption of the monolayer onto the surface of the substrate (or the coating, if present). When the substrate or coating is a-metal or metal alloy, X, at least prior to incorporation into the monolayer, can in one embodiment be chosen to be an asymmetrical or symmetrical disulfide, sulfide, diselenide, selenide, thiol, isonitrile, selenol, a trivalent phosphorus compound, isothiocyanate, isocyanate, xanthanate, thiocarbamate, a phosphine, an amine, thio acid or a dithio acid. This embodiment is especially preferred when a coating or substrate is used that is a noble metal such as gold, silver, or platinum.

If the substrate is a material such as silicon, silicon oxide, indium tin oxide, magnesium oxide, alumina, quartz, glass, or silica, then, in one embodiment, the biosensor may comprise an X that, prior to incorporation into said monolayer, is a monohalosilane, dihalosilane, trihalosilane, trialkoxysilane, dialkoxysilane, or a monoalkoxysilane. Among these silanes, trichlorosilane and trialkoxysilane are exemplary.

In certain embodiments, the substrate is selected from the group consisting of silicon, silicon dioxide, indium tin oxide, alumina, glass, and titania; and X is selected from the group consisting of a monohalosi lane, dihalosi lane, trihalosilane, trichlorosi lane, trialkoxysilane, dialkoxysilane, monoalkoxysilane, carboxylic acids, and phosphates.

In another embodiment, the substrate of the sensor is silicon and X is an olefin.

In still another embodiment, the coating (or the substrate if no coating is present) is titania or tantalum oxide and X is a phosphate.

In other embodiments, the surface of the substrate (or coating thereon) is composed of a material such as titanium oxide, tantalum oxide, indium tin oxide, magnesium oxide, or alumina where X is a carboxylic acid or alkylphosphoric acid. Alternatively, if the surface of the substrate (or coating thereon) of the sensor is copper, then X may optionally be a hydroxamic acid.

If the substrate used in the invention is a polymer, then in many cases a coating on the substrate such as a copper coating will be included in the sensor. An appropriate functional group X for the coating would then be chosen for use in the sensor. In an alternative embodiment comprising a polymer substrate, the surface of the polymer may be plasma-modified to expose desirable surface functionalities for monolayer formation. For instance, EP 780423 describes the use of a monolayer molecule that has an alkene X functionality on a plasma exposed surface. Still another possibility for the invention sensor comprised of a polymer is that the surface of the polymer on which the monolayer is formed is functionalized by copolymerization of appropriately functionalized precursor molecules.

Another possibility is that prior to incorporation into the monolayer, X can be a free-radical-producing moiety. This functional group is especially appropriate when the surface on which the monolayer is formed is a hydrogenated silicon surface. Possible free-radical producing moieties include, but are not limited to, diacylperoxides, peroxides, and azo compounds. Alternatively, unsaturated moieties such as unsubstituted alkenes, alkynes, cyano compounds and isonitrile compounds can be used for —X, if the reaction with X is accompanied by ultraviolet, infrared, visible, or microwave radiation.

In alternative embodiments, X may be a hydroxyl, carboxyl, vinyl, sulfonyl, phosphoryl, silicon hydride, or an amino group.

The component Y is a functional group responsible for binding a dye containing sensor onto the substrate. In one embodiment, the Y group is either highly reactive (activated) towards the dye containing sensor or is easily converted into such an activated form. In certain embodiments, the coupling of Y with the selectivity component of the biosensor occurs readily under normal physiological conditions. The functional group Y may either form a covalent linkage or a noncovalent linkage with the selectivity component of the biosensor. In other embodiments, the functional group Y forms a covalent linkage with the selectivity component of the biosensor. It is understood that following the attachment of the selectivity component of the biosensor to Y, the chemical nature of Y may have changed. Upon attachment of the biosensor, Y may even have been removed from the organic linker.

In one embodiment of the sensor of the present invention, Y is a functional group that is activated in situ. Possibilities for this type of functional group include, but are not limited to, such simple moieties such as a hydroxyl, carboxyl, amino, aldehyde, carbonyl, methyl, methylene, alkene, alkyne, carbonate, aryliodide, or a vinyl group. Appropriate modes of activation would be obvious to one skilled in the art. Alternatively, Y can comprise a functional group that requires photoactivation prior to becoming activated enough to trap the protein-capture agent.

In another embodiment, Y is a complex and highly reactive functional moiety that needs no in situ activation prior to reaction with the selectivity component of the biosensor. Such possibilities for Y include, but are not limited to, maleimide, N-hydroxysuccinimide (Wagner et al., Biophysical Journal, 1996, 70:2052-2066), nitrilotriacetic acid (U.S. Pat. No. 5,620,850), activated hydroxyl, haloacetyl, bromoacetyl, iodoacetyl, activated carboxyl, hydrazide, epoxy, aziridine, sulfonylchloride, trifluoromethyldiaziridine, pyridyldisulfide, N-acyl-imidazole, imidazolecarbamate, vinylsulfone, succinimidylcarbonate, arylazide, anhydride, diazoacetate, benzophenone, isothiocyanate, isocyanate, imidoester, fluorobenzene, and biotin.

In an alternative embodiment, the functional group Y is selected from the group of simple functional moieties. Possible Y functional groups include, but are not limited to —OH, —NH₂, —COOH, —COOR, —RSR, —PO₄⁻³, —OSO₃⁻², —SO₃⁻, —COO⁻, —SOO⁻, —CONR₂, —CN, —NR₂, and the like.

In another embodiment, one or more biosensor species may be bound to discrete beads or microspheres. The microspheres typically are either carboxylated or avidin-modified so that proteins, such as antibodies, non-antibody receptors and variants and fragments thereof, may be readily attached to the beads by standard chemistries. In an exemplary embodiment, the selectivity components are scFv fragments. The scFv fragments may be bound to carboxylated beads by one of many linking chemistries, such as, for example, EDC chemistry, or bound to avidin-coated beads by first biotinylating the scFv fragment by one of many common biotinylation chemistries, such as, for example, by conjugation with sulfo-NHS-LC-biotin (Pierce).

In another embodiment, two or more biosensors are affixed to one or more supports at discrete locations (that is, biosensors having a first specificity are affixed at a first spatial location, biosensors having a second specificity are affixed at a second spatial location, etc.). In one embodiment, the biosensors are affixed to a substrate in a tiled array, with each biosensor represented in one or more positions in the tiled array. The spatial configuration of the substrate or substrates may be varied so long as each biosensor species is bound at detectably discrete locations. The substrate and tiled biosensor pattern typically is planar, but may be any geometric configuration desired. For instance, the substrate may be a strip or cylindrical, as illustrated in U.S. Pat. No. 6,057,100, FIGS. 3A-3E. In exemplary embodiments, the substrate may be glass or other silicic compositions, such as those used in the semiconductor industry. Fabrication of the substrate may be by one of many well-known processes. In various embodiments, the biosensors of the array may be associated with the same reporter molecule or may be associated with different reporter molecules. Identification of a biosensor that interacts with a target molecule may be based on the signal from the reporter molecule, the location of the biosensor on the array, or a combination thereof. Arrays may be used in association with both the in vitro and in vivo applications of the invention.

In various embodiments, the arrays may comprise any of the biosensors described herein, including, for example, arrays of biosensors wherein the selectivity components are polypeptides (including antibodies and variants or fragments thereof), polynucleotides (i.e., aptamers), template imprinted materials, organic binding elements, and inorganic binding elements. The arrays may comprise one type of biosensor or a mixture of different types of biosensors (e.g., a mixture of biosensors having polypeptide and polynucleotide selectivity components). Protein microarrays are described, for example, in PCT Publication WO 00/04389, incorporated herein by reference. Examples of commercially available protein microarrays are those of Zyomyx of Hayward, California, Ciphergen Biosystems, Inc. of Fremont, Calif. and Nanogen, Inc. of San Diego, Calif. Nucleic acid microarrays are described, for example, in U.S. Pat. Nos. 6,261,776 and 5,837,832. Examples of commercially available nucleic acid microarrays are those of Affymetrix, Inc. of Santa Clara, Calif., BD Biosciences Clontech of Palo Alto, Calif. and Sigma-Aldrich Corp. of St. Louis, Mo.

4. Reporters

The reporter may be any molecule which produces a detectable signal change in response to an alteration in the environment. For example, the signal change may be an increase or decrease in signal intensity, or a change in the type of signal produced. In exemplary embodiments, suitable reporters include molecules which produce optically detectable signals, including, for example, fluorescent and chemiluminescent molecules. In certain embodiments, the reporter molecule is a long wavelength fluorescent molecule which permits detection of the reporter signal through a tissue sample, especially non-invasive detection of the reporter in conjunction with in vivo applications.

In certain embodiments, the reporter molecule is a pH sensitive fluorescent dye (pH sensor dye) which shows a spectral change upon interaction of a selectivity component with a target molecule. Interaction of the selectivity component with a target molecule may lead to a shift in the pH of the microenvironment surrounding the selectivity component due to the composition of acidic and basic residues on the selectivity and/or target molecules. In turn, the shift in the pH microenvironment leads to a detectable spectral change in the signal of the pH sensitive fluorescent dye molecule associated with the selectivity component. In exemplary embodiments, a pH sensitive dye is selected with an appropriate pKa to lead to an optimal spectral change upon binding of the particular selectivity component/target molecule combination. A variety of pH sensitive dyes suitable for use in accordance with the invention are commercially available. In exemplary embodiments, pH sensitive dyes include, for example, fluorescein, umbelliferones (coumarin compounds), pyrenes, resorufin, hydroxy esters, aromatic acids, styryl dyes, tetramethyl rhodamine dyes, and cyanine dyes, and pH sensitive derivatives of fluorescein, umbelliferones (coumarin compounds), pyrenes, resorufin, hydroxy esters, aromatic acids, styryl dyes, tetramethyl rhodamine dyes, and cyanine dyes.

In other embodiments, the reporter molecule is a polarity sensitive fluorescent dye (polarity sensor dye) which shows a spectral change upon interaction of a selectivity component with a target molecule. Interaction of the selectivity component with a target molecule may lead to a shift in the polarity of the microenvironment surrounding the selectivity component due to the composition of polar and/or non-polar residues on the selectivity and/or target molecules. In turn, the change in the polarity of the microenvironment leads to a detectable spectral change in the signal of the polarity sensitive fluorescent dye molecule associated with the selectivity component. A variety of polarity sensitive dyes suitable for use in accordance with the invention are commercially available. In exemplary embodiments, polarity sensitive dyes include, for example, merocyanine dyes, 5-((((2-iodoacetyl)amino)ethyl)amino)naphthalene-1-sulfonic acid (1,5-IAEDANS), and CPM, and polarity sensitive derivatives of merocyanine dyes, IAEDANS, and CPM.

In certain embodiments, the reporter molecule is a fluorescent dye that is sensitive to changes in the microviscosity of the local environment (restriction sensor dye). Interaction of the selectivity component with a target molecule may lead to a change in the microviscosity in the local environment surrounding the selectivity component. In turn, the change in microviscosity may lead to a detectable spectral change in the signal of the mobility sensor dye molecule associated with the selectivity component. For example, an increase of microviscosity upon target binding will restrict the dye and increase the quantum yield of the emitted fluorescence signal. A variety of restriction sensor dyes suitable for use in accordance with the invention are commercially available. In exemplary embodiments, restriction sensor dyes include, for example, monomethine and trimethine cyanine dyes, and microviscosity sensitive derivatives of monomethine and trimethine cyanine dyes.

In certain embodiments, the reporter molecule is a fluorescent dye that exhibits a spectral change due to a modification in the tumbling rate of the dye as measured on a nanosecond time scale (mobility sensor dye). Mobility sensor dye molecules may be linked to the selectivity component using a linker molecule that permits free rotation of the dye molecule. Upon interaction of the selectivity component with a target molecule, the rotation of the dye molecule around the linker may become restricted leading to a change in the ratio of parallel to perpendicular polarization of the dye molecule. A change in polarization of the mobility sensor dye may be detected as a change in the spectral emission of the dye and can be measured using light polarization optics for both excitation and emission to determine the tumbling rate of the dye. Abbott's fluorescence polarization technology is an exemplary method for determining the polarization of the dye. In exemplary embodiments, the mobility sensor dye is attached to the selectivity component using a triple-bond containing linker that extends the dye away from the surface of the selectivity component. A variety of mobility sensor dyes suitable for use in accordance with the invention are commercially available. In exemplary embodiments, mobility sensor dyes include, for example, cyanine dyes and derivatives thereof.

In certain embodiments, the reporter molecule is represented by structure I, II, or III: 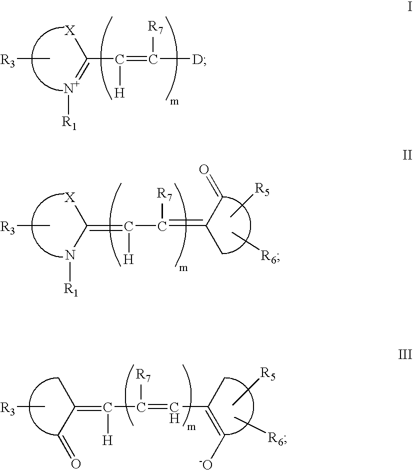
wherein:
the curved lines represent the atoms necessary to complete a structure selected from one ring, two fused rings, and three fused rings, each said ring having five or six atoms, and each said ring comprising carbon atoms and, optionally, no more than two atoms selected from oxygen, nitrogen and sulfur;
D is 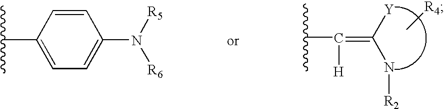
m is 1, 2, 3 or 4;
X and Y are independently selected from the group consisting of O, S, and —C(CH₃)₂—;
at least one R₁, R₂, R₃, R₄, R₅, R₆, or R₇ is a reactive group such as a group containing isothiocyanate, isocyanate, monochlorotriazine, dichlorotriazine, mono- or di-halogen substituted pyridine, mono- or di-halogen substituted diazine, phosphoramidite, maleimide, aziridine, sulfonyl halide, acid halide, hydroxysuccinimide ester, hydroxysulfosuccinimide ester, imido ester, hydrazine, axidonitrophenyl, azide, 3-(2-pyridyl dithio)-proprionamide, glyoxal, haloacetamido, or aldehyde;
providing that when any of R₁, R₂, R₃, R₄, R₅, R₆, or R₇ is not a reactive group it is selected from the group consisting of H, alkyl, aryl, and an -E-F group;
wherein:
   F is selected from the group consisting of hydroxy, protected hydroxy, alkoxy, sulfonate, sulfate, carboxylate, and lower alkyl substituted amino or quartenary amino;
   E is spacer group of formula —(CH₂)ₙ— wherein n is an integer from 0-5 inclusively;
   further providing that R₁ and R₂ may be joined by a —CHR₈—CHR₈— or —BF₂-biradical;
wherein;
   R₈ independently for each occurrence is selected from the group consisting of hydrogen, amino, quaternary amino, aldehyde, aryl, hydroxyl, phosphoryl, sulfhydryl, water solubilizing groups, alkyl groups of twenty-six carbons or less, lipid solubilizing groups, hydrocarbon solubilizing groups, groups promoting solubility in polar solvents, groups promoting solubility in nonpolar solvents, and -E-F; and
   further providing that any of R₁, R₂, R₃, R₄, R₅, R₆, or R₇ may be substituted with halo, nitro, cyano, —CO₂alkyl, —CO₂H, —CO₂aryl, NO₂, or alkoxy.

The following are more specific examples of reporter molecules according to structure I, II, or III: 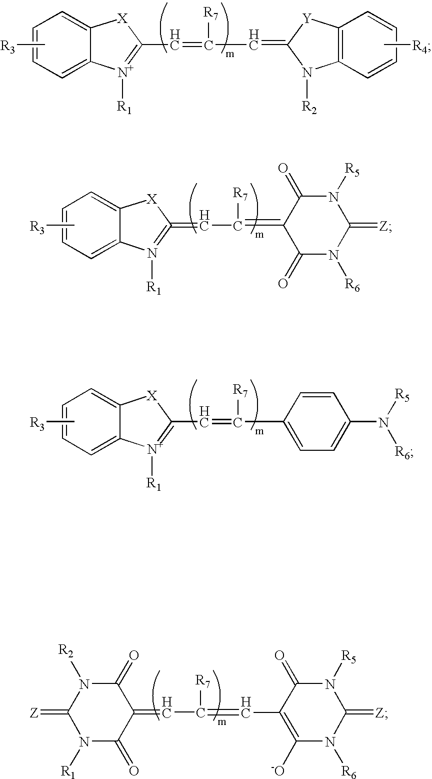
In these structures
X and Y are selected from the group consisting of O, S and —CH(CH₃)₂—;
Z is selected from the group consisting of O and S;
m is an integer selected from the group consisting of 1, 2, 3 and 4 and, preferably an integer from 1-3.

In the above formulas, the number of methine groups determines in part the excitation color. The cyclic azine structures can also determine in part the excitation color. Often, higher values of m contribute to increased luminescence and absorbance. At values of m above 4, the compound becomes unstable. Thereupon, further luminescence can be imparted by modifications at the ring structures. When m=2, the excitation wavelength is about 650 nm and the compound is very fluorescent. Maximum emission wavelengths are generally 15-100 nm greater than maximum excitation wavelengths.

The polymethine chain of the luminescent dyes of this invention may also contain one or more cyclic chemical groups that form bridges between two or more of the carbon atoms of the polymethine chain. These bridges might serve to increase the chemical or photostability of the dye and might be used to alter the absorption and emission wavelength of the dye or change its extinction coefficient or quantum yield. Improved solubility properties may be obtained by this modification.

In certain embodiments, the reporter molecule is represented by structure IV: 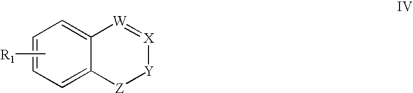
wherein:
W is N or C(R1);
X is C(R₂)₂;
Y is C(R₃)₂;
Z is NR₁, O, or S;
at least one R₁, R₂, or R₃ is a reactive group such as a group containing isothiocyanate, isocyanate, monochlorotriazine, dichlorotriazine, mono- or di-halogen substituted pyridine, mono- or di-halogen substituted diazine, phosphoramidite, maleimide, aziridine, sulfonyl halide, acid halide, hydroxysuccinimide ester, hydroxysulfosuccinimide ester, imido ester, hydrazine, axidonitrophenyl, azide, 3-(2-pyridyl dithio)-proprionamide, glyoxal or aldehyde;
providing that when any of R₁, R₂, or R₃ is not a reactive group it is selected from the group consisting of H; alkyl; aryl; 1, 2, or 3 fused rings, each said ring having five or six atoms, and each said ring comprising carbon atoms and, optionally, no more than two atoms selected from oxygen, nitrogen and sulfur; and an -E-F group;
wherein:
   F is selected from the group consisting of hydroxy, protected hydroxy, alkoxy, sulfonate, sulfate, carboxylate, and lower alkyl substituted amino or quartenary amino;
   E is spacer group of formula —(CH₂)ₙ— wherein n is an integer from 0-5 inclusively;
   further providing that two R₃ taken together may form O, S, NR₁, or N⁺(R₁)₂; or
      two R₃ along with R₂ may form 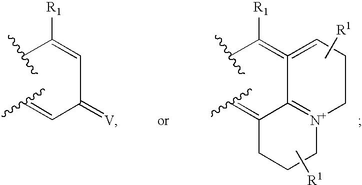
wherein V is O, S, NR₁, or N⁺(R₁)₂; and
further providing that any of R₁, R₂, or R₃ may be substituted with halo, nitro, cyano, —CO₂alkyl, —CO₂H, —CO₂aryl, NO₂, or alkoxy.

The following are more specific examples of reporter molecules according to structure IV:

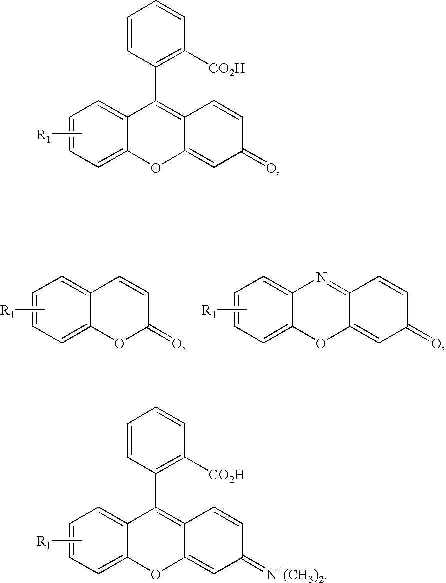

In certain embodiments, the reporter molecule is represented by structure V: 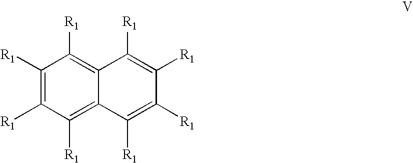
wherein:
at least one R₁ is a reactive group such as groups containing isothiocyanate, isocyanate, monochlorotriazine, dichlorotriazine, mono- or di-halogen substituted pyridine, mono- or di-halogen substituted diazine, phosphoramidite, maleimide, aziridine, sulfonyl halide, acid halide, hydroxysuccinimide ester, hydroxysulfosuccinimide ester, imido ester, hydrazine, axidonitrophenyl, azide, 3-(2-pyridyl dithio)-proprionamide, glyoxal, haloacetamido, or aldehyde;
providing that when any of R1 is not a reactive group it is selected from the group consisting of H, alkyl, aryl, and an -E-F group;
wherein:
   F is selected from the group consisting of hydroxy, protected hydroxy, alkoxy, sulfonate, sulfate, carboxylate, and lower alkyl substituted amino or quartenary amino;
   E is spacer group of formula —(CH₂)ₙ— wherein n is an integer from 0-5 inclusively;
   further providing that any two adjacent R1 may be joined to form a fused aromatic ring; and
   further providing that R1 may be substituted with halo, nitro, cyano, —CO₂alkyl, —CO₂H, —CO₂aryl, NO₂, or alkoxy.

The following are more specific examples of reporter molecules according to structure V:

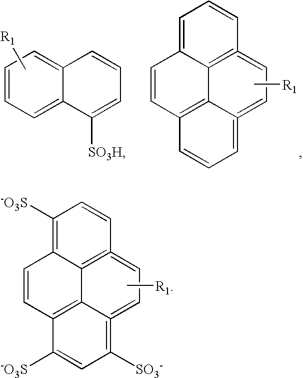

At least one, preferably only one, and possibly two or more of either R₁, R₂, R₃, R₄, R₅, R₆ and R₇ groups in each molecule is or contains a reactive group covalently reactive with amine, protected or unprotected hydroxy or sulfhydryl nucleophiles for attaching the dye to the labeled component. For certain reagents, at least one of said R₁, R₂, R₃, R₄, R₅, R₆ and R₇ groups on each molecule may also be a group that increases the solubility of the chromophore, or affects the selectivity of labeling of the labeled component or affects the position of labeling of the labeled component by the dye.

Reactive groups that may be attached directly or indirectly to the chromophore to form R₁, R₂, R₃, R₄, R₅, R₆ and R₇ groups may include reactive moieties such as groups containing isothiocyanate, isocyanate, monochlorotriazine, dichlorotriazine, mono- or di-halogen substituted pyridine, mono- or di-halogen substituted diazine, phosphoramidite, maleimide, aziridine, sulfonyl halide, acid halide, hydroxysuccinimide ester, hydroxysulfosuccinimide ester, imido ester, hydrazine, axidonitrophenyl, azide, 3-(2-pyridyl dithio)-proprionamide, glyoxal, haloacetamido, and aldehyde.

Specific examples of R₁, R₂, R₃, R₄, R₅, R₆ and R₇ groups that are especially useful for labeling components with available amino-, hydroxy-, and sulfhydryl groups include: 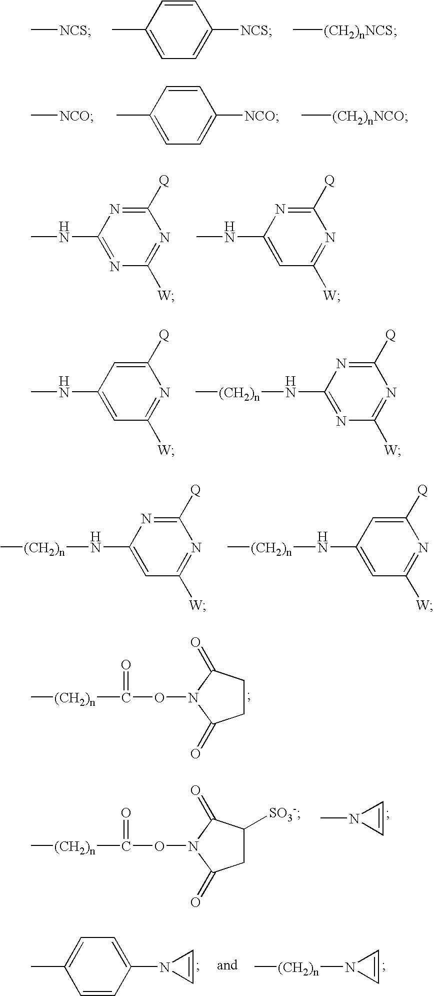
wherein at least one of Q or W is a leaving group such as I, Br or Cl.

Specific examples of R₁, R₂, R₃, R₄, R₅, R₆ and R₇ groups that are especially useful for labeling components with available sulfhydryls which can be used for labeling selectivity components in a two-step process are the following: 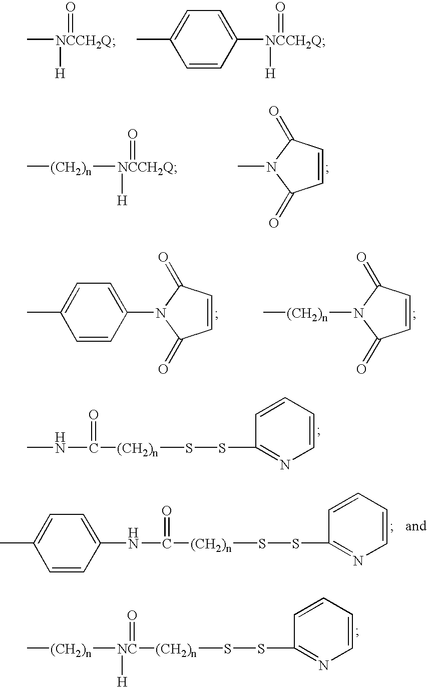
wherein Q is a leaving group such as I or Br, and wherein n is an integer.

Specific examples of R₁, R₂, R₃, R₄, R₅, R₆ and R₇ groups that are especially useful for labeling components by light-activated cross linking include:

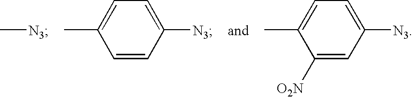

For the purpose of increasing water solubility or reducing unwanted nonspecific binding of the labeled component to inappropriate components in the sample or to reduce the interactions between two or more reactive chromophores on the labeled component which might lead to quenching of fluorescence, the R₁, R₂, R₃, R₄, R₅, R₆ and R₇ groups can be selected from the well known polar and electrically charged chemical groups.

In certain embodiments of the invention, the reporter molecule is represented by structure I, II, III, IV, or V and the accompanying definitions, and is a pH sensitive reporter molecule.

In certain embodiments of the invention, the reporter molecule is represented by structure I, II, III, IV or V and the accompanying definitions, and is a polarity sensitive reporter molecule.

In certain embodiments of the invention, the reporter molecule is represented by structure I, II, III, IV, or V and the accompanying definitions, and is a microviscosity reporter molecule.

In certain embodiments of the invention, the reporter molecule is represented by structure I, II, III, IV, or V and the accompanying definitions, and is a mobility sensor reporter molecule.

In various embodiments, the spectral change of the sensor dye upon interaction of the selectivity component and a target molecule may include, for example, a shift in absorption wavelength, a shift in emission wavelength, a change in quantum yield, a change in polarization of the dye molecule, and/or a change in fluorescence intensity. Any method suitable for detecting the spectral change associated with a given sensor dye may be used in accordance with the inventions. In exemplary embodiments, suitable instruments for detection of a sensor dye spectral change, include, for example, fluorescent spectrometers, filter fluorometers, microarray readers, optical fiber sensor readers, epifluorescence microscopes, confocal laser scanning microscopes, two photon excitation microscopes, and flow cytometers.

In various embodiments, the reporter molecule may be associated with the selectivity component or the target molecule. In exemplary embodiments, the reporter molecule is covalently attached to the selectivity component. The reporter molecule may be covalently attached to the selectivity component using standard techniques. In certain embodiments the reporter molecule may be directly attached to the selectivity component by forming a chemical bond between one or more reactive groups on the two molecules. In an exemplary embodiment, a thiol reactive reporter molecule is attached to a cysteine residue (or other thiol containing molecule) on the selectivity component. Alternatively, the reporter molecule may be attached to the selectivity component via an amino group on the selectivity component molecule. In other embodiments, the reporter molecule may be attached to the selectivity component via a linker group. Suitable linkers that may be used in accordance with the inventions include, for example, chemical groups, an amino acid or chain of two or more amino acids, a nucleotide or chain of two or more polynucleotides, polymer chains, and polysaccharides. In exemplary embodiments, the reporter molecule is attached to the selectivity component using a linker having a maleimide moiety.

In various embodiments, one or more reporter molecules may be attached at one or more locations on the selectivity component. For example, two or more molecules of the same reporter may be attached at different locations on a single selectivity component molecule. Alternatively, two or more different reporter molecules may be attached at different locations on a single selectivity component molecule. In exemplary embodiments, 2, 3, 4, 5, 6, 7, 8, 9, 10 or more reporter molecules are attached at different sites on the selectivity component. The one or more reporter molecules may be attached to the selectivity component so as to maintain the activity of the reporter molecule and the selectivity component. In certain embodiments, the location of the reporter molecule is optimized to permit exposure of the reporter molecule to changes in the microenvironment upon interaction of the selectivity component with a target molecule while maintaining the ability of the selectivity component to interact with the target molecule. In exemplary embodiments, the reporter molecule is attached to the selectivity component in spatial proximity to the target binding site without affecting the ability of the selectivity component to interact with the target molecule.

5. Exemplary Uses

The biosensors of the invention may be used to detect and/or quantitate analytes in any solid, liquid or gas sample. In various exemplary embodiments, the biosensors of the invention may be used for a variety of diagnostic and/or research applications, including, for example, monitoring the development of engineered tissues, in vivo monitoring of analytes of interest (including polynucleotides, polypeptides, hormones, lipids, carbohydrates, and small inorganic and organic compounds and drugs) using injectable free biosensors or implants functionalized with one or more biosensors, biological research (including developmental biology, cell biology, neurobiology, immunology, and physiology), detection of microbial, viral and botanical polynucleotides or polypeptides, drug discovery, medical diagnostic testing, environmental detection (including detection of hazardous substances/hazardous wastes, environmental pollutants, chemical and biological warfare agents, detection of agricultural diseases, pests and pesticides and space exploration), monitoring of food freshness and/or contamination, food additives, and food production and processing streams, monitoring chemical and biological products and contaminants, and monitoring industrial and chemical production and processing streams.

In one embodiment, the biosensors described herein may be used for detecting environmental pollutants, including, air, water and soil pollutants. Examples of air pollutants, include, for example, combustion contaminants such as carbon monoxide, carbon dioxide, nitrogen dioxide, sulfur dioxide, and tobacco smoke; biological contaminants such as animal dander, molds, mildew, viruses, pollen, dust mites, and bacteria; volatile organic compounds such as formaldehyde, fragrance products, pesticides, solvents, and cleaning agents; heavy metals such as lead or mercury; and asbestos, aerosols, ozone, radon, lead, nitrogen oxides, particulate matter, refrigerants, sulfur oxides, and volatile organic compounds. Examples of soil pollutants, include, for example, acetone, arsenic, barium, benzene, cadmium, chloroform, cyanide, lead, mercury, polychlorinated biphenyls (PCBs), tetrachloroethylene, toluene, and trichloroethylene (TCE). Examples of water pollutants, include, for example, arsenic, contaminated sediment, disinfection byproducts, dredged material, and microbial pathogens (e.g., Aeromonas, Coliphage, Cryptosporidium, E. coli, Enterococci, Giardia, total coliforms, viruses).

In other embodiments, the biosensors described herein may be used for detecting hazardous substances, including, for example, arsenic, lead, mercury, vinyl chloride, polychlorinated biphenyls (PCBs), benzene, cadmium, benzopyrene, polycyclic aromatic hydrocarbons, benzofluoranthene, chloroform, DDT, aroclors, trichloroethylene, dibenz[a,h]anthracene, dieldrin, hexavalent chromium, chlordane, hexachlorobutadiene, etc.

In another embodiment, the biosensors described herein may be used for detecting chemical and biological warfare agents. Examples of biological warfare agents, include, for example, bacteria such as anthrax (Bacillus anthracis), botulism (Clostridium botulinum toxin), plague (Yersinia pestis), tularemia (Francisella tullarensis), brucellosis (Brucella species), epsilon toxin from Clostridium perfringens, food safety threats (e.g., Salmonella species, Escherichia coli 0157:H7, Shigella), water safety threats (e.g., Vibrio cholerae and Cryptosporidium parvum), glanders (Burkholderia mallei), Melioidosis (Burkholderia pseudomallei), Psittacosis (Chlamydia psittaci), Q fever (Coxiella burnetii), Ricin toxin from Ricinus communis, Staphylococcal enterotoxin B, Typhus fever (Rickettsia prowazekii) and viruses such as filoviruses (e.g., ebola or Marburg), arenaviruses (e.g., Lassa and Machupo), hantavirus, smallpox (variola major), hemorrhagic fever virus, Nipah virus, and alphaviruses (e.g., Venezuelan equine encephalitis, eastern equine encephalitis, western equine encephalitis). Examples of chemical warfare agents, include for example, blister agents (e.g., distilled mustard, lewisite, mustard gas, nitrogen mustard, phosgene oxime, ethyldichloroarsine, methyldichloroarsine, phenodichloroarsine, sesqui mustard), blood poisoning agents (arsine, cyanogen chloride, hydrogen chloride, hydrogen cyanide), lung damaging agents (chlorine, diphosgene, cyanide, nitrogen oxide, perfluorisobutylene, phosgene, red phosphorous, sulfur trioxide-chlorosulfonic acid, teflon, titanium tetrachloride, zinc oxide), incapacitating agents (agent 15, BZ, canniboids, fentanyls, LSD, phenothiazines), nerve agents (cyclohexyl sarin, GE, Soman, Sarin, Tabun, VE, VG, V-Gas, VM, VX), riot control/tear gas agents (bromobenzylcyanide, chloroacetophenone, chloropicrin, CNB, CNC, CNS, CR, CS), and vomit-inducing agents (adamsite, diphenylchloroarsine, diphenylcyanoarsine).

In another embodiment, the biosensors described herein may be used for monitoring food freshness and/or contamination, food additives, and food production and processing streams. Examples of bacterial contaminants that may lead to foodborne illnesses include, for example, Bacillus anthracis, Bacillus cereus, Brucella abortus, Brucella melitensis, Brucella suis, Campylobacter jejuni, Clostridium botulinum, Clostridium perfringens, Enterohemorrhagic E. Coli (including E. coli 0157:H7 and other Shiga toxin-producing E. coli), Enterotoxigenic E. coli, Listeria monocytogenes, Salmonella, Shigella, Staphylococcus aureus, Vibrio cholerae, Vibrio parahaemolyticus, Vibrio vulnificus, Yersinia enterocolytica and Yersinia pseudotuberculosis. Examples of viral contaminants that may lead to foodborne illnesses include, for example, hepatitis A, norwalk-like viruses, rotavirus, astroviruses, calciviruses, adenoviruses, and parvoviruses. Examples of parasitic contaminants that may lead to foodborne illnesses include, for example, Cryptosporidium parvum, Cyclospora cayetanensis, Entamoeba histolytica, Giardia lamblia, Toxoplasma gondii, and Trichinella spiralis. Examples of non-infectious toxins or contaminants that may lead to foodborne illnesses include, for example, antimony, arsenic, cadmium, ciguatera toxin, copper, mercury, museinol, muscarine, psilocybin, coprius artemetaris, ibotenic acid, amanita, nitrites, pesticides (organophosphates or carbamates), tetrodotoxin, scombroid, shellfish toxins, sodium floride, thallium, tin, vomitoxin, and zinc.

In one embodiment, the biosensors described herein may be used for in vitro and/or in vivo monitoring of analytes of interest. The biosensors may be injected or otherwise administered to a patient as free molecules or may be immobilized onto a surface before introduction into a patient. When administered as free molecules, the biosensors may be used to detect analytes of interest in both interstitial spaces and inside cells. For detection of analytes inside of cells, the selectivity component may be modified, as described above, with a tag that facilitates translocation across cellular membranes. Alternatively, the selectivity components may be introduced into cells using liposome delivery methods or mechanical techniques such as direct injection or ballistic-based particle delivery systems (see for example, U.S. Pat. No. 6,110,490). In other embodiments, the biosensors may be immobilized onto a surface (including, for example, a bead, chip, plate, slide, strip, sheet, film, block, plug, medical device, surgical instrument, diagnostic instrument, drug delivery device, prosthetic implant or other structure) and then introduced into a patient, for example, by surgical implantation. In exemplary embodiments, the biosensors are immobilized onto the surface of an implant, such as an artificial or replacement organ, joint, bone, or other implant. The biosensors of the invention may also be immobilized onto particles, optical fibers, and polymer scaffolds used for tissue engineering. For example, one or more biosensors may be immobilized onto a fiber optic probe for precise positioning in a tissue. The fiber optic then provides the pathway for excitation light to the sensor tip and the fluorescence signal back to the photodetection system.

In each of the various embodiments of the invention, a single biosensor may be used for detection of a single target molecule or two or more biosensors may be used simultaneously for detection of two or more target molecules. For example, 2, 5, 10, 20, 50, 100, 1000, or more, different selectivity components may be used simultaneously for detection of multiple targets. When using multiple selectivity components simultaneously, each selectivity component may be attached to a different reporter molecule to permit individual detection of target binding to each selectivity component. Alternatively, a dual detection system may be used where two or more selectivity components may be attached to the same reporter molecule (for example, the same sensor dye) and be identified based on a second detectable signal. For example, selectivity components having different target specificities but containing the same sensor dye may be distinguished based on the signal from a color coded particle to which it is attached. The read out for each selectivity component involves detection of the signal from the sensor dye, indicating association with the target molecule, and detection of the signal from the color coded particle, permitting identification of the selectivity component. In an exemplary embodiment, a panel of biosensors may be attached to a variety of color coded particles to form a suspension array (Luminex Corporation, Austin, Tex.). The mixture of coded particles associated with the biosensors of the invention may be mixed with a biological sample or administered to a patient. Flow cytometry or microdialysis may then be used to measure the signal from the sensor dye and to detect the color code for each particle. In various embodiments, the identification signal may be from a color coded particle or a second reporter molecule, including, for example, chemiluminescent, fluorescent, or other optical molecules, affinity tags, and radioactive molecules.

In other embodiments, one or more biosensors of the invention may be immobilized onto a three dimensional surface suitable for implantation into a patient. The implant allows monitoring of one or more analytes of interest in a three dimensional space, such as, for example, the spaces between tissues in a patient.

In other embodiments, the biosensors of the invention may be exposed to a test sample. Any test sample suspected of containing the target may be used, including, but not limited to, tissue samples such as biopsy samples and biological fluids such as blood, sputum, urine and semen samples, bacterial cultures, soil samples, food samples, cell cultures, etc. The target may be of any origin, including animal, plant or microbiological (e.g., viral, prokaryotic, and eukaryotic organisms, including bacterial, protozoal, and fungal, etc.) depending on the particular purpose of the test. Examples include surgical specimens, specimens used for medical diagnostics, specimens used for genetic testing, environmental specimens, cell culture specimens, food specimens, dental specimens and veterinary specimens. The sample may be processed or purified prior to exposure to the biosensor(s) in accordance with techniques known or apparent to those skilled in the art.

In other embodiments, the biosensors of the invention may be used to detect bacteria and eucarya in food, beverages, water, pharmaceutical products, personal care products, dairy products or environmental samples. Preferred beverages include soda, bottled water, fruit juice, beer, wine or liquor products. The biosensors of the invention are also useful for the analysis of raw materials, equipment, products or processes used to manufacture or store food, beverages, water, pharmaceutical products, personal care products, dairy products or environmental samples.

Alternatively, the biosensors of the invention may be used to diagnose a condition of medical interest. For example the methods, kits and compositions of this invention will be particularly useful for the analysis of clinical specimens or equipment, fixtures or products used to treat humans or animals. In one preferred embodiment, the assay may be used to detect a target sequence which is specific for a genetically based disease or is specific for a predisposition to a genetically based disease. Non-limiting examples of diseases include, beta-Thalassemia, sickle cell anemia, Factor-V Leiden, cystic fibrosis and cancer related targets such as p53, p10, BRC-1 and BRC-2. In still another embodiment, the target sequence may be related to a chromosomal DNA, wherein the detection, identification or quantitation of the target sequence can be used in relation to forensic techniques such as prenatal screening, paternity testing, identity confirmation or crime investigation.

In still other embodiments, the methods of the invention include the analysis or manipulation of plants and genetic materials derived therefrom as well as bio-warfare reagents. Biosensors of the invention will also be useful in diagnostic applications, in screening compounds for leads which might exhibit therapeutic utility (e.g. drug development) or in screening samples for factors useful in monitoring patients for susceptibility to adverse drug interactions (e.g. pharmacogenomics).

In certain embodiments, the biosensors of the invention may be formulated into a pharmaceutical composition comprising one or more biosensors and a pharmaceutically acceptable carrier, adjuvant, or vehicle. The term “pharmaceutically acceptable carrier” refers to a carrier(s) that is “acceptable” in the sense of being compatible with the other ingredients of a composition and not deleterious to the recipient thereof. Methods of making and using such pharmaceutical compositions are also included in the invention. The pharmaceutical compositions of the invention can be administered orally, parenterally, by inhalation spray, topically, rectally, nasally, buccally, vaginally, or via an implanted reservoir. The term parenteral as used herein includes subcutaneous, intracutaneous, intravenous, intramuscular, intra articular, intrasynovial, intrastemal, intrathecal, intralesional, and intracranial injection or infusion techniques.

In other embodiments, the invention contemplates kits including one or more biosensors of the invention, and other subject materials, and optionally instructions for their use. Uses for such kits include, for example, environmental and/or biological monitoring or diagnostic applications.

6. Equivalents

The present invention provides among other things novel proteins, protein structures and protein-protein interactions. While specific embodiments of the subject invention have been discussed, the above specification is illustrative and not restrictive. Many variations of the invention will become apparent to those skilled in the art upon review of this specification. The appended claims are not intended to claim all such embodiments and variations, and the full scope of the invention should be determined by reference to the claims, along with their full scope of equivalents, and the specification, along with such variations.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

7. Incorporation by Reference

All publications and patents mentioned herein, including those items listed below, are hereby incorporated by reference in their entirety as if each individual publication or patent was specifically and individually indicated to be incorporated by reference. In case of conflict, the present application, including any definitions herein, will control.

Also incorporated by reference are the following: U.S. Pat. Nos. 5,334,537; 5,998,142; 6,287,765; 6,297,059; 6,331,394; 6,358,710; WO 02/23188; WO 02/18952; Bark and Hahn, Methods 20: 429-435 (2000); Barker et al., Anal. Chem. 71: 1767-1772 (1999); Barker et al., Anal. Chem. 71: 2071-2075 (1999); Bradbury, Nature Biotechnology 19: 528-529 (2001); Benhar, Biotechnology Advances 19: 1-33 (2001); Carrero and Voss, J. Biol. Chem. 271: 5332-5337 (1996); Chamberlain and Hahn, Traffic 1: 755-762 (2000); Chen et al., Nature Biotechnology 19: 537-542 (2001); Hahn et al., J. Biol. Chem. 265: 20335-20345 (1990); Marks et al., J. Mol. Biol. 222: 581-597 (1991); Post et al., J. Biol. Chem. 269: 12880-12887 (1994); Post et al., Mol. Biol. Cell 6: 1755-1768 (1995); Ramjiawan et al., Cancer 89: 1134-44 (2000); Skerra, J. Mol. Recognition 13: 167-187 (2000); and Sumner et al., Analyst 127: 11-16 (2002).

1. A biosensor comprising:
a selectivity component, and
at least one reporter molecule selected from the group consisting of a polarity sensor dye, a restriction sensor dye, and a mobility sensor dye,
wherein binding of the selectivity component to a target molecule produces a detectable change in the fluorescence signal of the reporter molecule.

2. The biosensor of claim 1, wherein the selectivity component is selected from the group consisting of a monoclonal antibody, polyclonal antibody, Fv fragment, single chain Fv (scFv) fragment, Fab′ fragment, F(ab′)2 fragment, single domain antibody, camelized antibody, humanized antibody, diabodies, tribodies, tetrabodies, aptamer, and template imprinted material.

3. The biosensor of claim 1, wherein the reporter molecule is represented by structure I: 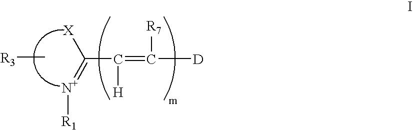
wherein:
the curved lines represent the atoms necessary to complete a structure selected from one ring, two fused rings, and three fused rings, each said ring having five or six atoms, and each said ring comprising carbon atoms and, optionally, no more than two atoms selected from oxygen, nitrogen and sulfur;
D is 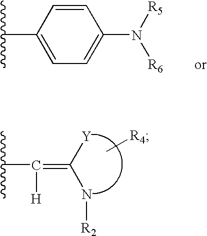
m is 1, 2, 3 or 4;
X and Y are independently selected from the group consisting of O, S, and —C(CH₃)₂—;
at least one R₁, R₂, R₃, R₄, R₅, R₆, or R₇ is a reactive group selected from the group consisting of isothiocyanate, isocyanate, monochlorotriazine, dichlorotriazine, mono- or di-halogen substituted pyridine, mono- or di-halogen substituted diazine, phosphoramidite, maleimide, aziridine, sulfonyl halide, acid halide, hydroxysuccinimide ester, hydroxysulfosuccinimide ester, imido ester, hydrazine, axidonitrophenyl, azide, 3-(2-pyridyl dithio)-proprionamide, glyoxal, haloacetamido, and aldehyde;
providing that when any of R₁, R₂, R₃, R4, R₅, R6, or R₇ is not a reactive group it is selected from the group consisting of H, alkyl, aryl, and an -E-F group;
wherein:
F is selected from the group consisting of hydroxy, protected hydroxy, alkoxy, sulfonate, sulfate, carboxylate, and lower alkyl substituted amino or quartenary amino;
E is spacer group of formula —(CH₂)ₙ— wherein n is an integer from 0-5 inclusively;
further providing that R₁ and R₂ may be joined by a —CHR₈—CHR₈— or —BF₂-biradical;
wherein;
R₈ independently for each occurrence is selected from the group consisting of hydrogen, amino, quaternary amino, aldehyde, aryl, hydroxyl, phosphoryl, sulfhydryl, water solubilizing groups, alkyl groups of twenty-six carbons or less, lipid solubilizing groups, hydrocarbon solubilizing groups, groups promoting solubility in polar solvents, groups promoting solubility in nonpolar solvents, and -E-F; and
further providing that any of R₁, R₂, R₃, R₄, R₅, R₆, or R₇ may be substituted with halo, nitro, cyano, —CO₂alkyl, —CO₂H, —CO₂aryl, NO₂, or alkoxy.

4-7. (canceled)

8. The biosensor of claim 3 wherein the reporter molecule is a restriction sensor dye.

9. The biosensor of claim 3 wherein the reporter molecule is a polarity sensor dye.

10. The biosensor of claim 3 wherein the reporter molecule is a mobility sensor dye.

11. The biosensor of claim 1, wherein the restriction sensor dye is a monomethine cyanine dye or a trimethine cyanine dye.

12. The biosensor of claim 1, wherein the dye is a polarity sensor dye.

13. The biosensor of claim 1, wherein the dye is a mobility sensor dye.

14. The biosensor of claim 1, wherein the reporter is fluorescent.

15. The biosensor of claim 1, wherein the biosensor further comprises a chemical handle.

16-28. (canceled)

29. The biosensor of claim 1, wherein the reporter is detectable using a fluorescent spectrometer, filter fluorometer, microarray reader, optical fiber sensor reader, epifluorescence microscope, confocal laser scanning microscope, two photon excitation microscope, or a flow cytometer.

30-36. (canceled)

37. A biosensor comprising:
a selectivity component that is selected from the group consisting of an Fv fragment, single chain Fv (scFv) fragment, Fab′ fragment, F(ab′)2 fragment, single domain antibody, camelized antibody, humanized antibody, diabodies, tribodies, tetrabodies, aptamer, and template imprinted material, and
at least one reporter molecule that is responsive to environmental changes,
wherein binding of the selectivity component to a target molecule provides a detectable change in the fluorescence signal of the reporter molecule.

38-46. (canceled)

47. The biosensor of claim 37, wherein the reporter molecule is a pH sensor dye.

48. The biosensor of claim 37, wherein the reporter molecule is a polarity sensor dye.

49. The biosensor of claim 37, wherein the reporter molecule is a restriction sensor dye.

50. The biosensor of claim 49, wherein the restriction sensor dye is a monomethine cyanine dye, a trimethine cyanine dye.

51. The biosensor of claim 37, wherein the reporter molecule is a mobility sensor dye.

52. (canceled)

53. A method for detecting at least one target molecule comprising:
providing at least one biosensor comprising a selectivity component and a reporter molecule; and
detecting the signal of the reporter molecule,
wherein interaction of the biosensor with the target molecule produces a detectable change in the fluorescence signal of the reporter molecule.

54-77. (canceled)

78. The method of claim 53 wherein the biosensor comprises a selectivity component selected from the group consisting of monoclonal antibody, polyclonal antibody, Fv fragment, single chain Fv (scFv) fragment, Fab′ fragment, F(ab′)2 fragment, single domain antibody, camelized antibody, humanized antibody, diabodies, tribodies, tetrabodies, aptamer, and template imprinted material.

79. (canceled)

80. The method of claim 78, wherein the reporter molecule is selected from the group consisting of a pH sensor dye, a polarity sensor dye, a restriction sensor dye, and a mobility sensor dye.

81-91. (canceled)
